(19) United States
(12) Patent Application Publication
(10) Pub. No.: US 2025/0034959 A1
(43) Pub. Date: Jan. 30, 2025

(54) APPARATUSES AND SYSTEMS FOR IN-LINE WATER ANALYSIS, DOWNHOLE FLUID OPTIMIZATION, AND METHODS FOR MAKING AND USING SAME

(71) Applicant: PfP INDUSTRIES LLC, Houston, TX (US)

(72) Inventors: Asoke Kumar Deysarkar, Houston, TX (US); Brian Decaires, Houston, TX (US); Nikhil Patel, Houston, TX (US); Christopher Dilley, Houston, TX (US)

(73) Assignee: PfP INDUSTRIES LLC, Houston, TX (US)

(21) Appl. No.: 18/765,040

(22) Filed: Jul. 5, 2024

Related U.S. Application Data
(60) Provisional application No. 63/525,107, filed on Jul. 5, 2023.

(51) Int. Cl. E21B 21/06; E21B 43/26; E21B 47/00; G01N 33/28

(52) U.S. Cl. CPC E21B 21/062; E21B 43/2607; E21B 47/00; G01N 33/2823

(57) ABSTRACT

A real-time or near real-time, downhole fluid formulation apparatuses and/or systems and methods of making and using same include (a) a water supply assembly/subsystem, (b) an in-line sensor assembly/subsystem, (c) a downhole fluid component supply assembly/subsystem, (d) a friction-reducing component supply assembly/subsystem, (e) a downhole fluid injection assembly/subsystem, and (f) a control assembly/subsystem, wherein (1) the in-line sensor assembly/subsystem generates data concerning various aqueous base fluid properties and (2) the downhole fluid component supply assembly/subsystem supplies downhole fluid components to the aqueous base fluid based on the aqueous base fluid properties and various formation properties, and the friction-reducing component supply assembly/subsystem supplies friction-reducing components to form a downhole fluid having a reduced or minimized percent drag reduction (% DR) value.

### RELATED APPLICATIONS

This application claims the benefit of and priority to U.S. Provisional Patent Application Ser. No. 63/525,107 filed Jul. 5, 2023 (5 Jul. 2023).

### BACKGROUND OF THE DISCLOSURE

### 1. Field of the Disclosure

Embodiments of the present disclosure relate to real-time or near real-time, downhole fluid formulation adjustment apparatuses and/or systems and methods of making and using same.

In particular, embodiments of the present disclosure relate to real-time or near real-time, downhole fluid formulation adjustment apparatuses and/or systems and methods of making and using same, wherein the real-time or near real-time, downhole fluid formulation adjustment apparatuses and/or systems include (a) an aqueous base fluid supply assembly/subsystem, (b1) a stand-still in-line aqueous fluid sensor assembly/subsystem or (b2) a flowing in-line aqueous fluid sensor assembly/subsystem, (c) a downhole sensor assembly/subsystem, (d) a downhole fluid component supply assembly/subsystem, (c) a friction-reducing component supply assembly/subsystem, a (f1) a downhole fluid injection assembly/subsystem or (f2) a downhole circulation assembly/subsystem, and (g) a control assembly/subsystem.

### 2. Description of the Related Art

Drilling fluid are used to drill oil and/or gas wells, and treating fluids are used in a variety of subterranean treatment operations for a variety of different results after an oil and/or gas well has been drilled into an oil and/or gas bearing subterranean formation or formations or zones or intervals thereof. One subterranean treatment operation is hydraulic fracturing operations, which generally involves pumping a fracturing fluid or a hydraulic fracturing into a wellbore that penetrates a subterranean oil and/or gas bearing formation or formations under hydraulic fracturing conditions to create or enhance one or more cracks or fractures in the subterranean oil and/or gas bearing formation or formations or zones or intervals thereof. The fracturing fluid may include particulates, often referred to as proppant particulates or simply proppants, that are deposited in the fractures. The proppant functions, inter alia, to prevent the fractures or cracks from fully closing after release of hydraulic pressure, forming conductive channels through which fluids and hydrocarbons may flow into the wellbore for transport to the surface.

In one such approach, a slickwater hydraulic fracturing may be used. A slickwater hydraulic fracturing is a fracturing fluid that does not include a cross-linked polymer and has a relatively low viscosity as a result. Slickwater fracturing fluids may be used to generate narrow, complex fractures or cracks with low proppants concentrations and still remain open after fracturing is completed. Because the viscosity of the slickwater fracturing fluid is relatively low, the proppant transport is achieved by increasing the pumping rate and pressure of the slickwater fracturing fluid. During pumping, significant energy loss often occurs due to friction between the slickwater fracturing fluid and the casing or tubing, particularly when the slickwater fracturing fluid is in a state of turbulent flow.

Friction reducing compositions are often introduced into the slickwater fracturing fluids during fracturing operations to minimize such energy loss due to fluid friction during fracturing operations. The friction reducing compositions are typically uncross-linked polymer-containing compositions as cross-linking polymer-containing compositions often reduce friction reduction during pumping operations, and in fact, often results in increased friction during pumping operations. The friction reducing compositions facilitate laminar flow of the slickwater fracturing fluids, which cause less frictional forces and energy loss than turbulent flow of the same fluid.

Currently, there are no apparatuses, systems, or methods implementing them for on-the-fly or in real-time or near real-time, in-line water analysis to allow real-time or near real-time modification of friction reducing composition type and amount being added to a treating fluid to minimize frictional drag as the treating fluid being pumped from the surface through a tubular mechanism into a subterranean oil and/or gas bearing formation, formations, portions, zones and/or intervals thereof.

Thus, there is still a need in the art for apparatuses, systems, or methods implementing them to on-the-fly or in real-time or near real-time, in-line water to allow real-time or near real-time modification of friction reducing composition type and amount being added to a treating fluid to minimize frictional drag as the treating fluid being pumped from the surface through a tubular mechanism into a subterranean oil and/or gas bearing formation, formations, portions, zones and/or intervals thereof.

### SUMMARY OF THE DISCLOSURE

### Apparatuses

Embodiments of this disclosure provide real-time or near real-time, downhole fluid formulation adjustment apparatuses including: (a) an aqueous base fluid supply assembly, (b) a stand-still in-line aqueous fluid sensor assembly, (c) a downhole sensor assembly, (d) a downhole fluid component supply assembly, (c) a friction-reducing component supply assembly, (f) a downhole fluid injection assembly, and (g) a control assembly.

Embodiments of this disclosure provide real-time or near real-time, downhole fluid formulation adjustment apparatuses including: (a) an aqueous base fluid supply assembly, (b) a stand-still in-line aqueous fluid sensor assembly, (c) a downhole sensor assembly, (d) a downhole fluid component supply assembly, (c) a friction-reducing component supply assembly, (f) a downhole circulation assembly, and (g) a control assembly.

Embodiments of this disclosure provide real-time or near real-time, downhole fluid formulation adjustment apparatuses including: (a) an aqueous base fluid supply assembly, (b) a flowing in-line aqueous fluid sensor assembly, (c) a downhole sensor assembly, (d) a downhole fluid component supply assembly, (c) a friction-reducing component supply assembly, (f) a downhole fluid injection assembly, and (g) a control assembly.

Embodiments of this disclosure provide real-time or near real-time, downhole fluid formulation adjustment apparatuses including: (a) an aqueous base fluid supply assembly, (b) a flowing in-line aqueous fluid sensor assembly, (c) a downhole sensor assembly, (d) a downhole fluid component supply assembly, (c) a friction-reducing component supply assembly, (f) a downhole circulation assembly, and (g) a control assembly.

The stand-still or flowing in-line aqueous fluid sensor assembly is configured to generate real-time or near real-time data concerning a composition and properties of the aqueous base fluid and downhole sensor assembly is configured to generate real-time or near real-time data concerning a composition and properties of the downhole fluid and real-time or near real-time data concerning the treating or drilling properties.

The downhole fluid component supply assembly is configured to supply downhole fluid components to the aqueous base fluid based on the data to form a downhole fluid and the friction-reducing component supply assembly supplies friction-reducing components to the downhole fluid based on the data to form an optimized downhole fluid having a reduced or minimized percent drag reduction (% DR) value. In certain embodiments, the downhole sensor assembly may also include a program configured to generate modeling data for adjusting a composition and properties of the optimized downhole fluid based on the sensor and modeling data.

The control assembly is configured to utilize the data to adjust amount of the downhole fluid components, the friction-reducing components being added to the aqueous base fluid in real-time or near real-time.

### Systems

Embodiments of this disclosure provide real-time or near real-time, downhole fluid formulation adjustment systems including: (a) an aqueous base fluid supply subsystem, (b) a stand-still in-line aqueous fluid sensor subsystem, (c) a downhole sensor subsystem, (d) a downhole fluid component supply subsystem, (c) a friction-reducing component supply subsystem, (f) a downhole fluid injection subsystem, and (g) a control subsystem.

Embodiments of this disclosure provide real-time or near real-time, downhole fluid formulation adjustment systems including: (a) an aqueous base fluid supply subsystem, (b) a stand-still in-line aqueous fluid sensor subsystem, (c) a downhole sensor subsystem, (d) a downhole fluid component supply subsystem, (c) a friction-reducing component supply subsystem, (f) a downhole circulation subsystem, and (g) a control subsystem.

Embodiments of this disclosure provide real-time or near real-time, downhole fluid formulation adjustment systems including: (a) an aqueous base fluid supply subsystem, (b) a flowing in-line aqueous fluid sensor subsystem, (c) a downhole sensor subsystem, (d) a downhole fluid component supply subsystem, (c) a friction-reducing component supply subsystem, (f) a downhole fluid injection subsystem, and (g) a control subsystem.

Embodiments of this disclosure provide real-time or near real-time, downhole fluid formulation adjustment systems including: (a) an aqueous base fluid supply subsystem, (b) a flowing in-line aqueous fluid sensor subsystem, (c) a downhole sensor subsystem, (d) a downhole fluid component supply subsystem, (c) a friction-reducing component supply subsystem, (f) a downhole circulation subsystem, and (g) a control subsystem.

The stand-still or flowing in-line aqueous fluid sensor subsystem is configured to generate real-time or near real-time data concerning a composition and properties of the aqueous base fluid and downhole sensor subsystem is configured to generate real-time or near real-time data concerning a composition and properties of the downhole fluid and real-time or near real-time data concerning the treating or drilling properties.

The downhole fluid component supply subsystem is configured to supply downhole fluid components to the aqueous base fluid based on the data to form a downhole fluid and the friction-reducing component supply subsystem supplies friction-reducing components to the downhole fluid based on the data to form an optimized downhole fluid having a reduced or minimized percent drag reduction (% DR) value. In certain embodiments, the downhole sensor subsystem may also include a program configured to generate modeling data for adjusting a composition and properties of the optimized downhole fluid based on the sensor and modeling data.

The control subsystem is configured to utilize the data to adjust amount of the downhole fluid components, the friction-reducing components being added to the aqueous base fluid in real-time or near real-time.

### Methods of Treating

Embodiments of this disclosure provide methods, implemented in an apparatus or system as set forth above, comprising: (a) supplying an aqueous base fluid from an aqueous based fluid supply assembly/subsystem, (b) passing the aqueous base fluid through a flowing or stand-still, in-line aqueous base fluid sensor assembly/subsystem, (c) receiving aqueous fluid data from the flowing or stand-still, in-line aqueous base fluid sensor assembly/subsystem via the control assembly/subsystem, (d) receiving downhole data via the control assembly/subsystem, (c) adding amounts of downhole fluid components to the aqueous base fluid based the aqueous fluid data via a downhole treating fluid component supply assembly/subsystem to form a downhole treating fluid, (f) adding amounts of friction-reducing components to the downhole fluid via a friction-reducing component supply assembly/subsystem to form an optimized downhole treating fluid, (g) injecting the optimized downhole treating fluid via a downhole fluid injection assembly/subsystem into a subterranean formation, (h) controlling the adding steps and the injecting step via a control assembly/subsystem, and (i) repeating the above step until the treating operation stops.

The stand-still or flowing in-line aqueous fluid sensor subsystem is configured to generate real-time or near real-time data concerning a composition and properties of the aqueous base fluid and downhole sensor subsystem is configured to generate real-time or near real-time data concerning a composition and properties of the downhole treating fluid and real-time or near real-time data concerning the treating properties.

The downhole fluid component supply subsystem is configured to supply downhole fluid components to the aqueous base fluid based on the data to form a downhole treating fluid and the friction-reducing component supply subsystem supplies friction-reducing components to the downhole treating fluid based on the data to form an optimized downhole treating fluid having a reduced or minimized percent drag reduction (% DR) value. In certain embodiments, the downhole sensor subsystem may also include a program configured to generate modeling data for adjusting a composition and properties of the optimized downhole treating fluid based on the sensor and modeling data.

The control subsystem is configured to utilize the data to adjust amount of the downhole treating fluid components, the friction-reducing components being added to the aqueous base fluid in real-time or near real-time.

### Methods of Drilling

Embodiments of this disclosure provide real-time or near real-time, downhole fluid formulation methods including: (a) supplying an aqueous base fluid from an aqueous based fluid supply assembly/subsystem, (b) passing the aqueous base fluid through a flowing or stand-still, in-line aqueous base fluid sensor assembly/subsystem, (c) receiving aqueous fluid data from the flowing or stand-still, in-line aqueous base fluid sensor assembly/subsystem via the control assembly/subsystem, (d) receiving downhole data via the control assembly/subsystem, (c) adding amounts of downhole fluid components to the aqueous base fluid based the aqueous fluid data via a downhole fluid component supply assembly/subsystem to form a downhole drilling fluid, (f) adding amounts of friction-reducing components to the downhole fluid via a friction-reducing component supply assembly/subsystem to form an optimized downhole drilling fluid, (g) circulating the optimized downhole drilling fluid via a downhole fluid circulating assembly/subsystem in a wellbore to a subterranean formation, (h) controlling the adding steps and the circulating step via a control assembly/subsystem, and (i) repeating the above step until the drilling operation stops.

The stand-still or flowing in-line aqueous fluid sensor subsystem is configured to generate real-time or near real-time data concerning a composition and properties of the aqueous base fluid and downhole sensor subsystem is configured to generate real-time or near real-time data concerning a composition and properties of the downhole drilling fluid and real-time or near real-time data concerning the drilling properties.

The downhole fluid component supply subsystem is configured to supply downhole fluid components to the aqueous base fluid based on the data to form a downhole fluid and the friction-reducing component supply subsystem supplies friction-reducing components to the downhole drilling fluid based on the data to form an optimized downhole drilling fluid having a reduced or minimized percent drag reduction (% DR) value. In certain embodiments, the downhole sensor subsystem may also include a program configured to generate modeling data for adjusting a composition and properties of the optimized downhole fluid based on the sensor and modeling data.

The control subsystem is configured to utilize the data to adjust amount of the downhole fluid components, the friction-reducing components being added to the aqueous base fluid in real-time or near real-time.

### BRIEF DESCRIPTION OF THE DRAWINGS OF THE DISCLOSURE

The disclosure may be better understood with reference to the following detailed description together with the appended illustrative drawings in which like elements are numbered the same:

FIG. 1A depicts an embodiment of an apparatus/system of this disclosure.

FIG. 1B depicts another embodiment of an apparatus/system of this disclosure.

FIGS. 2A-C depict embodiments of an in-line water analysis assembly/subsystem of this disclosure.

FIGS. 2D-F depict other embodiments of an in-line water analysis assembly/subsystem of this disclosure.

FIG. 3 depicts an embodiment of a downhole fluid component supply subsystem of this disclosure.

FIG. 4 depicts an embodiment of a friction-reducing component supply assembly/subsystem of this disclosure.

FIGS. 5A&B depict embodiments of downhole fluid injection assemblies/subsystem of this disclosure.

FIG. 6 depicts an embodiment of a control assembly/subsystem of this disclosure.

FIG. 7A depicts an embodiment of a flow diagram of a method for forming and injecting an optimized treating fluid into a formation to be treated via a tubular injection mechanism.

FIG. 7B depicts an embodiment of flow diagram of a method of drilling and circulating an optimized drilling fluid through a drilling string while drilling.

### DEFINITIONS USED IN THE DISCLOSURE

All terms used in this disclose and in the attached claims will be given their plain, ordinary meaning unless otherwise explicitly and clearly defined below:

The term “at least one”, “one or more”, or “one or a plurality” are interchangeable within this disclosure and refers to one item or more than one items, e.g., at least one polymer, one or more polymers, or one or a plurality of polymers means one polymer or more than one polymers. While these are open ended terms, one of ordinary skill in the art will understand in the context of the terms being used that there are practical limitations to the opened endedness of the terms. Generally, the upper limit is less than or equal to about 20, sometimes less than or equal to about 15, sometimes less than or equal to about 10, or sometimes less than or equal to about 5.

The term “about” or “approximately” refers to the fact that a value of a given quantity is within ±20% of the stated value, or within ±15% of the stated value, or within ±10% of the stated value, or within ±5% of the stated value, or within ±2.5% of the stated value, or within ±1% of the stated value.

The term “substantially” or “essentially” refers to the fact that a value of a given quantity is within ±5% of the stated value, or within ±2.5% of the stated value, or within ±2% of the stated value, or within ±1% of the stated value, or within ±0.1% of the stated value, or within ±0.01% of the stated value.

In this disclosure, every range of values (e.g., “from about x to about y” or “from approximately x to y” or “from approximately x-y”-“between about x and about y” or “between approximate x and y” or “between approximately x-y) is to be understood as referring to the ranges including end points and all subranges between x and y, e.g., between about X and Y includes all ranges x and y, where x is greater than X and y is less than Y.

The term “gpt” or “gptg” refers to gallons per thousand gallons.

The term “pptg” or “ppt” refers to pounds per thousand gallons.

The term “ppg” refers to pounds of particulates per gallon of treatment fluid.

The term “wt. %” refers to weight percent.

The term “w/w” refers to weight per weight.

The term “vol. %” refers to volume percent.

The term “v/v” refers to volume per volume.

The term “w/v” refers to weight per volume.

The term “v/w” refers to volume per weight.

The term “ppm” means parts per million.

The term “cps” or “cP” means centipoise.

The term “rpm” means revolutions per minute.

The term “TDS” means total dissolved solids and means waters having from about 60 ppm to about 120,000 ppm total dissolved solids.

The term “RO” means reverse osmosis.

The term “FR” means friction reducer or friction reducing agents.

The terms “drilling” and grammatical equivalents thereof refer to any downhole fluid for drilling wells of any type into a subterranean formation-oil, gas, water, gases, etc., or any combination thereof.

The terms “treat,” “treatment,” “treating,” and grammatical equivalents thereof refer to any subterranean oil and/or gas bearing formation treating operation that uses a downhole fluid to achieve a desired outcome. Use of these terms does not imply any particular action by a given downhole fluid.

The term “fracturing” refers to the process and methods of breaking down a subterranean oil and/or gas bearing formation surrounding a well bore, by pumping a downhole fluid at very high pressure into the subterranean oil and/or gas bearing formation to create fractures and increase production rates from the subterranean oil and/or gas bearing formation. The fracturing methods of this disclosure may use conventional techniques known in the art.

The term “under treating conditions” refers injecting or pumping a downhole fluid into a subterranean oil and/or gas bearing formation at a sufficient pressure, at a sufficient temperature (normally not an issue), and for a time sufficient to achieve a desired increase production rates from the subterranean oil and/or gas bearing formation.

The term “under fracturing conditions” refers injecting or pumping a fracturing fluid into a subterranean oil and/or gas bearing formation at a sufficient pressure, at a sufficient temperature (normally not an issue), and for a time sufficient to achieve a desired increase production rates from the subterranean oil and/or gas bearing formation.

The term “cracks”, “microcracks”, “fissures”, “microfissures”, “fractures”, or “microfactures” refers to create or enhance openings in the formation, where the term micro refers to smaller openings in the formation. Under fracturing conditions, the enhanced or created openings of fractures will generally have an elongated profile.

The term “proppant” and grammatical equivalents thereof refers to a granular substance suspended in the fracturing fluid during the fracturing operation, which serves to keep the formation from closing back down upon itself once the pressure is released. Proppants envisioned by the present disclosure include, but are not limited to, conventional proppants familiar to those skilled in the art such as sand, 20-40 mesh sand, resin-coated sand, sintered bauxite, glass beads, particular plant materials, and other solid materials using a proppant in fracturing operations.

The term “friction reducing (FR) polymers” refers to polymers used to reduce friction of a fracturing fluid as it is pumped through fracturing mechanisms into the formation to be fractured or to reduce frictional losses due to friction between an aqueous fluid in turbulent flow and tubular goods (e.g., pipes, coiled tubing, etc.) and/or the formation.

The term “FR base fluid” refers to the major component of the WB-FR slurry compositions of this disclosure (as opposed to components dissolved and/or suspended therein), and does not indicate any particular condition or property of that fluid such as its mass, amount, pH, etc.

The term “polymer” or “polymeric material” means or includes homopolymers, copolymers, terpolymers, etc.

The term “copolymer,” as used herein, means polymers including two or more monomers or monomeric units, e.g., terpolymers, tetrapolymers, etc.

The term “aqueous base fluid” refers to base fluids used in the water-based friction reducing additive of the present disclosure may include water from any source, which may include fresh water, saltwater (e.g., water containing one or more salts dissolved therein), brine (e.g., saturated saltwater), seawater, produced water, surface water (e.g., from a river or a pond), reclaimed water, any other water useable in downhole operations, or any combination thereof.

The term “real-time” means that the processing unit receives information from the sensors and immediately acts on the received information.

The term “near real-time” means that the processing unit receives information from the sensors and after a finite delay acts on the received information. The finite delay may be about 1 millisecond (10⁻³ seconds), about 1 centisecond (10⁻² seconds), about 1 tenth of a second (10⁻¹ seconds), one second, or greater than one second depending on operator election.

While embodiments of this disclosure have been depicted, such embodiments do not imply a limitation on the disclosure, and no such limitation should be inferred. The subject matter disclosed is capable of considerable modification, alteration, and equivalents in form and function, as will occur to those skilled in the pertinent art and having the benefit of this disclosure. The depicted and described embodiments of this disclosure are examples only and are not exhaustive of the scope of the disclosure.

### DETAILED DESCRIPTION OF THE DISCLOSURE

The inventors have found that real-time or near real-time, downhole fluid formulation apparatuses and/or systems and methods of making and using same may be constructed and/or implemented to control a downhole fluid composition and an introduction rate of one or more downhole fluids into a subterranean formation.

### Apparatuses

Embodiments of this disclosure provide broadly relates to apparatuses including: (c) an aqueous base fluid supply assembly, (d) a stand-still in-line aqueous fluid sensor assembly, (c) a downhole sensor assembly, (f) a downhole fluid component supply assembly, (g) a friction-reducing component supply assembly, (h) a downhole fluid injection assembly, and (i) a control assembly.

Embodiments of this disclosure provide broadly relates to apparatuses including: (c) an aqueous base fluid supply assembly, (d) a stand-still in-line aqueous fluid sensor assembly, (c) a downhole sensor assembly, (f) a downhole fluid component supply assembly, (g) a friction-reducing component supply assembly, (h) a downhole circulation assembly, and (i) a control assembly.

Embodiments of this disclosure provide broadly relates to apparatuses including: (c) an aqueous base fluid supply assembly, (d) a flowing in-line aqueous fluid sensor assembly, (c) a downhole sensor assembly, (f) a downhole fluid component supply assembly, (g) a friction-reducing component supply assembly, (h) a downhole fluid injection assembly, and (i) a control assembly.

Embodiments of this disclosure provide broadly relates to apparatuses including: (c) an aqueous base fluid supply assembly, (d) a flowing in-line aqueous fluid sensor assembly, (c) a downhole sensor assembly, (f) a downhole fluid component supply assembly, (g) a friction-reducing component supply assembly, (h) a downhole circulation assembly, and (i) a control assembly.

The stand-still or flowing in-line aqueous fluid sensor assembly is configured to generate real-time or near real-time data concerning a composition and properties of the aqueous base fluid and downhole sensor assembly is configured to generate real-time or near real-time data concerning a composition and properties of the downhole fluid and real-time or near real-time data concerning the treating or drilling properties.

The downhole fluid component supply assembly is configured to supply downhole fluid components to the aqueous base fluid based on the data to form a downhole fluid and the friction-reducing component supply assembly supplies friction-reducing components to the downhole fluid based on the data to form an optimized downhole fluid having a reduced or minimized percent drag reduction (% DR) value. In certain embodiments, the downhole sensor assembly may also include a program configured to generate modeling data for adjusting a composition and properties of the optimized downhole fluid based on the sensor and modeling data.

The control assembly is configured to utilize the data to adjust amount of the downhole fluid components, the friction-reducing components being added to the aqueous base fluid in real-time or near real-time.

### Systems

Embodiments of this disclosure provide broadly relates to systems including: (c) an aqueous base fluid supply subsystem, (d) a stand-still in-line aqueous fluid sensor subsystem, (c) a downhole sensor subsystem, (f) a downhole fluid component supply subsystem, (g) a friction-reducing component supply subsystem, (h) a downhole fluid injection subsystem, and (i) a control subsystem.

Embodiments of this disclosure provide broadly relates to systems including: (c) an aqueous base fluid supply subsystem, (d) a stand-still in-line aqueous fluid sensor subsystem, (e) a downhole sensor subsystem, (f) a downhole fluid component supply subsystem, (g) a friction-reducing component supply subsystem, (h) a downhole circulation subsystem, and (i) a control subsystem.

Embodiments of this disclosure provide broadly relates to systems including: (c) an aqueous base fluid supply subsystem, (d) a flowing in-line aqueous fluid sensor subsystem, (e) a downhole sensor subsystem, (f) a downhole fluid component supply subsystem, (g) a friction-reducing component supply subsystem, (h) a downhole fluid injection subsystem, and (i) a control subsystem.

Embodiments of this disclosure provide broadly relates to systems including: (c) an aqueous base fluid supply subsystem, (d) a flowing in-line aqueous fluid sensor subsystem, (e) a downhole sensor subsystem, (f) a downhole fluid component supply subsystem, (g) a friction-reducing component supply subsystem, (h) a downhole circulation subsystem, and (i) a control subsystem.

The stand-still or flowing in-line aqueous fluid sensor subsystem is configured to generate real-time or near real-time data concerning a composition and properties of the aqueous base fluid and downhole sensor subsystem is configured to generate real-time or near real-time data concerning a composition and properties of the downhole fluid and real-time or near real-time data concerning the treating or drilling properties.

The downhole fluid component supply subsystem is configured to supply downhole fluid components to the aqueous base fluid based on the data to form a downhole fluid and the friction-reducing component supply subsystem supplies friction-reducing components to the downhole fluid based on the data to form an optimized downhole fluid having a reduced or minimized percent drag reduction (% DR) value. In certain embodiments, the downhole sensor subsystem may also include a program configured to generate modeling data for adjusting a composition and properties of the optimized downhole fluid based on the sensor and modeling data.

The control subsystem is configured to utilize the data to adjust amount of the downhole fluid components, the friction-reducing components being added to the aqueous base fluid in real-time or near real-time.

### Methods of Treating

Embodiments of this disclosure broadly relates to methods, implemented in an apparatus or system as set forth above, comprising: (c) supplying an aqueous base fluid from an aqueous based fluid supply assembly/subsystem, (d) passing the aqueous base fluid through a flowing or stand-still, in-line aqueous base fluid sensor assembly/subsystem, (c) receiving aqueous fluid data from the flowing or stand-still, in-line aqueous base fluid sensor assembly/subsystem via the control assembly/subsystem, (f) receiving downhole data via the control assembly/subsystem, (g) adding amounts of downhole fluid components to the aqueous base fluid based the aqueous fluid data via a downhole treating fluid component supply assembly/subsystem to form a downhole treating fluid, (h) adding amounts of friction-reducing components to the downhole fluid via a friction-reducing component supply assembly/subsystem to form an optimized downhole treating fluid, (i) injecting the optimized downhole treating fluid via a downhole fluid injection assembly/subsystem into a subterranean formation, (j) controlling the adding steps and the injecting step via a control assembly/subsystem, and (k) repeating the above step until the treating operation stops.

The stand-still or flowing in-line aqueous fluid sensor subsystem is configured to generate real-time or near real-time data concerning a composition and properties of the aqueous base fluid and downhole sensor subsystem is configured to generate real-time or near real-time data concerning a composition and properties of the downhole treating fluid and real-time or near real-time data concerning the treating properties.

The downhole fluid component supply subsystem is configured to supply downhole fluid components to the aqueous base fluid based on the data to form a downhole treating fluid and the friction-reducing component supply subsystem supplies friction-reducing components to the downhole treating fluid based on the data to form an optimized downhole treating fluid having a reduced or minimized percent drag reduction (% DR) value. In certain embodiments, the downhole sensor subsystem may also include a program configured to generate modeling data for adjusting a composition and properties of the optimized downhole treating fluid based on the sensor and modeling data.

The control subsystem is configured to utilize the data to adjust amount of the downhole treating fluid components, the friction-reducing components being added to the aqueous base fluid in real-time or near real-time.

### Methods of Drilling

Embodiments of this disclosure broadly relates to real-time or near real-time, downhole fluid formulation methods including: (c) supplying an aqueous base fluid from an aqueous based fluid supply assembly/subsystem, (d) passing the aqueous base fluid through a flowing or stand-still, in-line aqueous base fluid sensor assembly/subsystem, (c) receiving aqueous fluid data from the flowing or stand-still, in-line aqueous base fluid sensor assembly/subsystem via the control assembly/subsystem, (f) receiving downhole data via the control assembly/subsystem, (g) adding amounts of downhole fluid components to the aqueous base fluid based the aqueous fluid data via a downhole fluid component supply assembly/subsystem to form a downhole drilling fluid, (h) adding amounts of friction-reducing components to the downhole fluid via a friction-reducing component supply assembly/subsystem to form an optimized downhole drilling fluid, (i) circulating the optimized downhole drilling fluid via a downhole fluid circulating assembly/subsystem in a wellbore to a subterranean formation, (j) controlling the adding steps and the circulating step via a control assembly/subsystem, and (k) repeating the above step until the drilling operation stops.

The stand-still or flowing in-line aqueous fluid sensor subsystem is configured to generate real-time or near real-time data concerning a composition and properties of the aqueous base fluid and downhole sensor subsystem is configured to generate real-time or near real-time data concerning a composition and properties of the downhole drilling fluid and real-time or near real-time data concerning the drilling properties.

The downhole fluid component supply subsystem is configured to supply downhole fluid components to the aqueous base fluid based on the data to form a downhole fluid and the friction-reducing component supply subsystem supplies friction-reducing components to the downhole drilling fluid based on the data to form an optimized downhole drilling fluid having a reduced or minimized percent drag reduction (% DR) value. In certain embodiments, the downhole sensor subsystem may also include a program configured to generate modeling data for adjusting a composition and properties of the optimized downhole fluid based on the sensor and modeling data.

The control subsystem is configured to utilize the data to adjust amount of the downhole fluid components, the friction-reducing components being added to the aqueous base fluid in real-time or near real-time.

Embodiments of this disclosure broadly relate to water supply assemblies/subsystems including one or more water sources to form a downhole aqueous base fluid.

Embodiments of this disclosure broadly relate to in-line sensor assemblies/subsystems including a plurality of sensors, wherein each of the sensors is designed to sense a given property of the downhole aqueous base fluid.

Embodiments of this disclosure broadly relate to downhole fluid component supply assemblies/subsystems including a plurality of downhole fluid component supply reservoirs, a plurality of control valves, and a plurality of conduits for supplying downhole fluid components to the aqueous downhole base fluid to form a first optimized downhole fluid.

Embodiments of this disclosure broadly relate to friction-reducing component supply assemblies/subsystems including a plurality of friction-reducing component supply reservoirs, a plurality of control valves, and a plurality of conduits for supplying friction-reducing components to the aqueous downhole base fluid or to the first optimized downhole fluid to form a second optimized downhole fluid.

Embodiments of this disclosure broadly relate to downhole fluid injection assemblies/subsystems including one or more optimized downhole fluid holding reservoirs, an injection assembly, and a tubular injection mechanism for directing the one or more optimized downhole fluids into a subterranean formation.

Embodiments of this disclosure broadly relate to downhole fluid circulation assemblies/subsystems including one or more optimized downhole fluid holding reservoirs, a circulation assembly, and a drilling equipment for drilling a well using one or more optimized downhole fluids.

Embodiments of this disclosure broadly relate to control assemblies/subsystems including a processing unit, memory, communication hardware and software, and software encoding a real-time or near real-time analysis of water properties. The control assemblies/subsystems further including software encoding a downhole fluid component addition amount and rate and a friction-reducing component addition amount and rate to formulate one or more optimized downhole fluids. The control assemblies/subsystems further including software encoding a circulation amount and rate or an injection amount and rate of the one or more optimized downhole fluids for drilling or treating a subterranean formation depending on the nature of the downhole fluid. The control assemblies/subsystems further including bi-directional communication pathways from the processing unit to control valves associated with the various assemblies/subsystems, where the pathway are designed to receive information/data from the sensors and to send information/data to the various assemblies/subsystems and to utilize the information/data to produce the one or more optimized downhole fluids.

The compositions, systems, and methods of this disclosure are designed to overcome the problems associated with using high TDS (total dissolved solids) waters including produced and/or flow back water and/or disposal water in fracturing fluids, which will allow the use and reuse of treated and untreated produced water with or without the addition of fresh water. The inventors have found that, contrary to existing knowledge and practices, cross-linked hydratable polymer based fracturing fluids may be produced using produced, flow back, and/or fracturing flow back water or mixtures of these waters and fresh water. In certain embodiments, the hydratable polymers are guar and/or guar derivatives and the cross-linking composition comprises a borate cross-linkers or combinations of borate crosslinkers or crosslinkers that are compatible with low pH approaches to crosslinking. To that end, we re-engineered the cross-linking process and designed buffers to handle the severity of high TDS fluids such as produced and/or flow back water and/or disposal water. One such process includes the following steps: (a) adding a first buffer to an aqueous base fluid including a high TDS water to lower a pH of the aqueous base fluid to form a low pH base fluid; (b) adding a hydratable polymer or hydratable polymer slurry to the base fluid to form a fracturing fluid; (c) adding a cross-linking composition to the fracturing fluid to form a pre-cross-linked fracturing fluid; and optionally (if the crosslinked system being utilized requires high pH) (d) adding a second buffer to increase the pH of the pre-cross-linked fracturing fluid to form a crosslinked fracturing fluid.

In certain embodiments, the first buffer is added to the base fluid at a level sufficient to adjust a pH of the base fluid to an acidic pH. In certain embodiments, the acidic pH is less than or equal to pH 7. In other embodiments, the acidic pH is less than or equal to pH 6. In other embodiments, the acidic pH is less than or equal to pH 5. In other embodiments, the acidic pH is less than or equal to pH 4. The buffer may include an inorganic acid, an organic acid, or mixtures thereof. The first buffer is added until the pH is at an acidic level for example at or below pH 7, 6, 5, or 4. In certain embodiments, an amount of the added first buffer is between about 0.1 gpt and about 5 gpt depending on the initial pH of the base fluid.

In certain embodiments where a high pH is needed, a second buffer is added to the fracturing fluid after the polymer or the polymer slurry and the crosslinking composition have been added at a level sufficient to adjust a pH of the fracturing fluid to at or above about pH 9. The second buffer may include an inorganic base, an organic base, or mixtures thereof. The second buffer is added until the pH is at or above about pH 9. In certain embodiments, an amount of the added second buffer is between about 0.1 gpt and about 5 gpt depending on the initial pH of the fracturing fluid. As the pH of the fracturing fluid prior to addition of the second buffer is considerably above about pH 9, cross-linking of the fracturing fluid does not start to occur until the pH of the fluid approaches pH 9 or greater. Thus, the compositions have a build in cross-link delay system, which is one of the unique features of the compositions, systems, and/or methods of this disclosure.

The compositions, systems and methods also provide systems that incorporate a crosslinked polymer approach based on a final pH that is below pH 7. In such systems, it may not be necessary to make a final pH adjustment (after the addition of the polymer or the polymer slurry and the crosslinking composition) to a pH at or above about pH 9. Still other compositions, systems and methods will provide approaches in which the crosslinked compositions are based on reduced polymer concentration levels of <10 ppt to achieve a controlled viscosity that will result in the generation of the desired long narrow type fractures, but with superior proppant transport as compared to low viscosity slick water designs and therefore increased propped fracture length.

Embodiments of the present disclosure broadly relates to fracturing fluid compositions including: (a) a base fluid comprising an elevated total dissolved solids (TDS) water selected from the group consisting to produced, flow back water, brackish water, reverse osmosis (RO) reject water, clear brine, and mixtures and combinations thereof, (b) a first buffer, (c) a hydratable polymer or a hydratable polymer slurry, and (d) a cross-linking composition, wherein the first buffer is adapted to adjust a pH of the base fluid to an acidic pH prior to adding the hydratable polymer or the hydratable polymer slurry and the cross-linking composition to the base fluid to reduce or prevent pre-mature polymer crosslinking, and wherein the cross-linking composition crosslinks the hydratable polymer after hydration to form the fracturing fluid composition having a crosslinked structure. In certain embodiments, the compositions may also include (e) a second buffer to adjust the pH to a final pH sufficient to activate the crosslinking composition toe form the crosslinked structure in the composition. In other embodiments, the second buffer adjusts the pH to at or above 9. In other embodiments, the base fluid further includes fresh water. In other embodiments, the hydratable polymer is selected from the group consisting of galactomannan gums, glucomannan gums, guars, derivatized guars, cellulose derivatives, synthetic polymers such as polyvinyl alcohol, polyacrylamides, poly-2-amino-2-methyl propane sulfonic acid, other synthetic polymers and copolymers, and mixtures or combinations thereof. In other embodiments, the hydratable polymer is a guar or derivatized guar and is in powder form when added to the composition or to the slurry. In other embodiments, the hydratable polymer is a mixture of a guar or derivatized guar and a polyacrylamide in which the polyacrylamide is present in an amount between 5 wt. % and 20 wt. % of the total polymer weight, and the polyacrylamide reduces a drag of the composition being pumped through a tubular downhole injection mechanism into a formation to be fracture and improves a shear stability of the crosslinked structure formed in the composition. In other embodiments, the hydratable polymer is present in an amount between about 0.005 wt. % and about 0.5 wt. % in the composition or if in a slurry form, the polymer is present in an amount between about 1 gpt and about 10 gpt in the slurry. In other embodiments, the amount is between about 0.005 wt. % and about 0.10 wt. % in the composition or between about 1 gpt and 2 gpt in the slurry. In other embodiments, the acidic pH is less than or equal to pH 7, or less than or equal to pH 6, or less than or equal to pH 5, or less than or equal to pH 4. In other embodiments, the crosslinking composition is capable of producing the crosslinked structure at the acidic pH. In other embodiments, the crosslinking composition comprises compounds including boron ions, zirconium ions, and titanium ions, or mixtures thereof. In other embodiments, the crosslinking composition comprises one or more borate compounds.

Embodiments of the present disclosure broadly relates to methods of fracturing a subterranean formation including pumping a fracturing fluid compositions including (a) a base fluid comprising an elevated total dissolved solids (TDS) water selected from the group consisting to produced, flow back water, brackish water, reverse osmosis (RO) reject water, clear brine, and mixtures and combinations thereof, (b) a first buffer, (c) a hydratable polymer or a hydratable polymer slurry, and (d) a cross-linking composition, wherein the first buffer is adapted to adjust a pH of the base fluid to an acidic pH prior to adding the hydratable polymer or the hydratable polymer slurry and the cross-linking composition so that the polymer may hydrate but not crosslink, and wherein the cross-linking composition crosslinks the hydratable polymer to form the fracturing fluid composition having a crosslinked structure. In certain embodiments, the composition used in the methods may also include a second buffer to adjust the pH to a final pH sufficient to activate the crosslinking composition. In other embodiments, the second buffer adjusts the pH to at or above about pH 9. In other embodiments, the base fluid further includes fresh water. In other embodiments, the methods may further include adding a proppant to the fracturing fluid being pumped into the well. In other embodiments, the hydratable polymer can be taken from a group that includes galactomannan gums, glucomannan gums, guars, derivatized guars, cellulose derivatives, synthetic polymers such as polyvinyl alcohol, polyacrylamides, poly-2-amino-2-methyl propane sulfonic acid, and various other synthetic polymers and copolymers and mixtures or combinations thereof. In other embodiments, the hydratable polymer is a guar or derivatized guar and is in powder form when added to the composition or to the slurry. In other embodiments, the hydratable polymer is a mixture of a guar or derivatized guar and a polyacrylamide in which the polyacrylamide is present in an amount between 5 wt. % and 20 wt. % of the total polymer weight, and the polyacrylamide reduces a drag of the composition being pumped through a tubular downhole injection mechanism into a formation to be fracture and improves a shear stability of the crosslinked structure formed in the composition In other embodiments, the hydratable polymer is present in an amount between about 0.005 wt. % and about 0.5 wt. % in the composition or if in a slurry form, the polymer is present in an amount between about 1 gpt and about 10 gpt in the slurry. In other embodiments, the amount is between about 0.005 wt. % and about 0.10 wt. % in the composition or between about 1 gpt and 2 gpt in the slurry. In other embodiments, the acidic pH is less than or equal to pH 7, or less than or equal to pH 6, or less than or equal to pH 5, or less than or equal to pH 4. In other embodiments, the crosslinking composition is capable of producing the crosslinked structure at the acidic pH. In other embodiments, the crosslinking composition is comprised of metal ions including boron, zirconium, and titanium containing compounds, or mixtures thereof. In other embodiments, the crosslinking composition comprises one or more borate compounds.

Increasing the polymer loading also allows the oppositely charged, water soluble, polyacrylamide containing polymer slurries of the present disclosure to utilize lower relative volumes while achieving the same performance as traditional emulsions. To illustrate, traditional emulsions are typically used at loadings between about 0.50 gpt and about 1.0 gpt in water. Aqueous concentrates require large volumes up to about 8 gpt as well as they comprise mostly water. Slurries of the present disclosure may be used at much lower concentration such as between about 0.1 gpt and about 0.5 gpt, while still providing improved drag reduction performance as compared to traditional emulsions, dry powders, and aqueous liquid concentrates.

Additionally, known emulsions are based on high molecular weight polymers, for example polymers having molecular weight of 24 million or higher. Slurries of the present disclosure may utilize lower molecular weight polymers, yet still give comparable drag reduction performance, even at lower loadings. Drag reduction and proppant delivery are the two primary functions of a friction reducer carrier fluid. Using particle size to increase polymer loading also increases the hydration viscosity and the downhole viscosity creating better proppant carrying properties.

The slurries of the present disclosure may also be modified to reduce free fluid in the final manufactured product by adding one or more synthetic polymeric suspending agents to oil-based carriers. In other embodiments, the oil-based carriers may include one or more organophilic clays. Traditional emulsions cannot be modified in this way. The addition of the one or more one or more synthetic polymeric suspending agents overcomes potential problems with free fluid and settling, resulting in a more stable product than a traditional emulsion. Alternatively, additional surfactants or adjustments in particle size may be used to reduce free fluid and settling. The reader is referred to United States Published application No. 20170313930A1 for additional information on slurries that may be used to formulate for use in this disclosure.

The present disclosure broadly relates to a composition for treatment of subterranean well formations comprising the slurry of as set forth above and a water source selected from the group consisting of fresh water, brackish water, salt water, sea water, produced water, flowback water, or combinations thereof; provided the selected water includes a sufficient concentration of ions to reduce gelling interactions of the oppositely charged polymers. In certain embodiments, the composition further comprising a proppant. In certain embodiments, the proppant is sand.

The present disclosure broadly relates to a method of treating a subterranean formation comprising hydrating an oil-based slurry and a water source selected from the group consisting of fresh water, brackish water, salt water, sea water, produced water, flowback water, or combinations thereof; provided the selected water includes a sufficient concentration of ions to reduce gelling interactions of the oppositely charged polymers to form a proppant carrier fluid; adding a proppant to the water source; and delivering the carrier fluid and proppant to the subterranean formation.

Embodiments of this disclosure broadly relates to slurry compositions including a mixture of oppositely charged, particulate, water-soluble polymers and an oil-based vehicle or carrier including one or more hydrocarbon solvents, one or more suspending agents, and one or more surfactants.

### Suitable Components for Use in the Disclosure

### Sensors

Suitable sensors for use in this disclosure include, without limitation, any sensor that measure a property of water such as salinity sensors, conductivity sensors, pH sensors, or other water properties or specific ion or chemical sensors that measure the concentration of specific ions or chemicals such as sodium ion sensors, potassium ion sensors, carbonate ion sensors, hydronium ion sensors, hydroxide ion sensors, borate ion sensors, carbon dioxide sensors, oxygen sensors, calcium ion sensors, magnesium ion sensors, titanium ion sensors, zirconium ion sensors, other ion specific sensors, or other chemical specific sensors, or any combination thereof.

### Aqueous Base Fluid Properties

Suitable aqueous base fluid properties include, without limitation, salinity, conductivity, pH, specific metal ions and/or metal salts, concentrations of the specific metal ions and/or metal salts, other ions and/or chemicals, ionicity, any other property of the aqueous base fluid, or any combination thereof.

### Downhole Fluid Properties

Suitable formation properties include, without limitation, formation temperature or temperature profile, formation pressure or pressure profile, formation geological structural properties, e.g., type of rock, shale, sand, etc., type and nature of natural fractures within the formation, extent of the formation to be treated, depth of penetration of the treating fluid, desired treating results, type of proppants to be used, type of proppant pillar formation, type of pumping format, pumping conditions such as pumping pressure, downhole fluid flow rate, pumping sequences, etc., other formation properties, or any combination thereof.

### Injection and Circulation Equipment

Suitable injection equipment and circulation equipment include, without limitation, any injecting and circulating systems used in the art.

### Polyacrylamide

Suitable acrylamide containing polymers or polyacrylamide containing polymers include, without limitation, polymers including acrylamide as a major monomer making up the polymer backbone. Generally, the term major in this setting means the polymers include at least 30% acrylamide, at least 40% acrylamide, at least 40% acrylamide, at least 50% acrylamide, at least 60% acrylamide, at least 70% acrylamide, at least 80% acrylamide, at least 90% acrylamide, or 100% acrylamide. It should be recognized that these ranges include all subranges such as 30% to 100% or any other range or any other at least percentage.

### Aqueous Base Fluids

Suitable aqueous base fluids include, without limitation, a high TDS produced water, a high TDS flow back water, a high TDS fracturing flow back water, a brackish water, a reverse osmosis (RO) reject water, a clear brine, and mixtures thereof. In certain embodiments, the aqueous base fluids further include fresh water.

### Oil-Based Base Fluids

Suitable oil-based base fluids include, without limitation, a hydrocarbon fluid such as diesel, kerosene, fuel oil, selected crude oils, a mineral oil, or any combination thereof.

### Hydratable Polymers

Suitable hydratable polymers or gelling agents that may be used in the disclosure include, without limitation, any hydratable polysaccharides that are capable of forming a gel in the presence of a crosslinking agent. Exemplary examples of hydratable polysaccharides include, without limitation, galactomannan gums, glucomannan gums, guars, derivatized guars, cellulose derivatives, and mixtures or combinations thereof. Specific examples are guar gum, guar gum derivatives, locust bean gum, Karaya gum, carboxymethyl cellulose, carboxymethyl hydroxyethyl cellulose, and hydroxyethyl cellulose. Other specific examples include, without limitation, guar gums, hydroxypropyl guar, carboxymethyl hydroxypropyl guar, carboxymethyl guar, and carboxymethyl hydroxyethyl cellulose. Suitable hydratable polymers may also include synthetic polymers, such as polyvinyl alcohol, polyacrylamides, poly-2-amino-2-methyl propane sulfonic acid, and various other synthetic polymers and copolymers. In certain embodiments, the molecular weight of the hydratable synthetic polymers are between about 10,000 to about 100,000,000. In other embodiments, the molecular weight is between about 10,000 to about 10,000,000. In other embodiments, the molecular weight is between about 10,000 to about 1,000,000.

The hydratable polymer may be present in a fracturing fluid in concentrations ranging from about 0.05 wt. % to about 10 wt. %. In certain embodiments, the polymer concentration ranges between about 0.10 wt. % and about 5.0 wt. %. In other embodiments, the polymer concentration ranges between about 0.05 w. % and about 0.7 wt. % of the aqueous fluid. In certain embodiments, the hydratable polymer is present in a range from about 0.10 wt. % to about 0.25 wtl. %. If the polymer is in the form or a slurry, then the slurry is present in an amount between about 10 gpt and about 30 gpt (gallons per thousand gallons) of the fracturing fluid. In certain embodiments, the polymer slurry amount is between about 1 gpt and about 15 gpt. In other embodiments, the polymer slurry amount is between about between about 2 gpt and about 5 gpt.

### Crosslinking Agents

Suitable crosslinking agents include, without limitation, any compound that increases the viscosity of a fluid including the hydratable polymers by chemical crosslinks, physical crosslinks, and/or cross-links the hydratable polymer by any other mechanism. For example, the gelation of a hydratable polymer may be achieved by cross-linking the polymer with metal ions including boron, zirconium, and titanium containing compounds, or mixtures thereof. One class of suitable crosslinking agents is organotitanates. Another class of suitable crosslinking agents is borates. The selection of an appropriate crosslinking agent depends upon the type of treatment to be performed and the hydratable polymer to be used. The amount of the crosslinking agent used also depends upon the well conditions and the type of treatment to be introduced. However, the range is generally from about 10 ppm to about 1000 ppm of metal ion of the crosslinking agent in the hydratable polymer fluid.

Other crosslinking agents may be a borate-containing compounds, titanate-containing compounds, zirconium-containing compound, and mixtures thereof. For example, the crosslinking agent can be sodium borate×H₂O (varying waters of hydration), boric acid, borate crosslinkers (a mixture of a titanate constituent, preferably an organotitanate constituent, with a boron constituent. The organotitanate constituent can be TYZOR R titanium chelate esters from E.I du Pont de Nemours & Company. The organotitanate constituent can be a mixture of a first organotitanate compound having a lactate base and a second organotitanate compound having triethanolamine base. The boron constituent can be selected from the group consisting of boric acid, sodium tetraborate, and mixtures thereof. These are described in U.S. Pat. No. 4,514,309, incorporated herein by reference, borate based ores such as ulexite and colemanite, Ti(IV) acetylacetonate, Ti(IV) triethanolamine, Zr lactate, Zr triethanolamine, Zr lactate-triethanolamine, Zr lactate-triethanolamine-triisopropanolamine, or mixtures thereof. In some embodiments, the well treatment fluid composition may further comprise a proppant.

Yet other crosslinking agents that crosslink polymer to even higher viscosities and more effective at carrying proppant into the fractured formation. The borate ion has been used extensively as a crosslinking agent, typically in high pH fluids, for guar, guar derivatives and other galactomannans. Sec, for example, U.S. Pat. No. 3,059,909, incorporated herein by reference and numerous other patents that describe this classic aqueous gel as a fracture fluid. Other crosslinking agents include, for example, titanium crosslinkers (U.S. Pat. No. 3,888,312, incorporated herein by reference), chromium, iron, aluminum, and zirconium (U.S. Pat. No. 3,301,723, incorporated herein by reference). Of these, the titanium and zirconium crosslinking agents are typically preferred. Examples of commonly used zirconium crosslinking agents include zirconium triethanolamine complexes, zirconium acetylacetonate, zirconium lactate, zirconium carbonate, and chelants of organic alphahydroxycorboxylic acid and zirconium. Examples of commonly used titanium crosslinking agents include titanium triethanolamine complexes, titanium acetylacetonate, titanium lactate, and chelants of organic alphahydroxycorboxylic acid and titanium. The crosslinking compositions may include mixtures or combination of any of crosslinking agents disclosed herein.

### Proppants

Suitable propping agents or proppants are typically added to the fracturing fluid prior to the addition of a crosslinking agent. However, proppants may be introduced in any manner which achieves the desired result. Any proppant may be used in embodiments of the disclosure. Examples of suitable proppants include, but are not limited to, quartz sand grains, glass and ceramic beads, walnut shell fragments, aluminum pellets, nylon pellets, and the like. Proppants are typically used in concentrations between about 1 lb to about 8 lbs. per gallon of a fracturing fluid, although higher or lower concentrations may also be used as desired. The fracturing fluid may also contain other additives, such as surfactants, corrosion inhibitors, mutual solvents, stabilizers, paraffin inhibitors, tracers to monitor fluid flow back, and so on.

### Breaking Agents

The term “breaking agent” or “breaker” refers to any chemical that is capable of reducing the viscosity of a gelled or crosslinked fluid. As described above, after a fracturing fluid is formed and pumped into a subterranean formation, it is generally desirable to convert the highly viscous gel to a lower viscosity fluid. This allows the fluid to be easily and effectively removed from the formation and to allow desired material, such as oil or gas, to flow through the proppant packed fracture into the well bore. This reduction in viscosity of the treating fluid is commonly referred to as “breaking”. The reduction in viscosity may be attributable to breaking the crosslinked structure, degrading the base polymer being used or a combination of both mechanisms. Consequently, the chemicals used to break the viscosity of the fluid is referred to as a breaking agent or a breaker.

Examples of inorganic breaking agents for use in this invention include, but are not limited to, persulfates, percarbonates, perborates, peroxides, perphosphates, permanganates, etc. Specific examples of inorganic breaking agents include, but are not limited to, alkaline earth metal persulfates, alkaline earth metal percarbonates, alkaline earth metal perborates, alkaline earth metal peroxides, alkaline earth metal perphosphates, zinc salts of peroxide, perphosphate, perborate, and percarbonate, and so on. Additional suitable breaking agents are disclosed in U.S. Pat. Nos. 5,877,127; 5,649,596; 5,669,447; 5,624,886; 5,106,518; 6,162,766; and 5,807,812, incorporated herein by reference. In some embodiments, an inorganic breaking agent is selected from alkaline earth metal or transition metal-based oxidizing agents, such as magnesium peroxides, zinc peroxides, and calcium peroxides.

In addition, enzymatic breakers may also be used in place of or in addition to a non-enzymatic breaker. Examples of suitable enzymatic breakers such as guar specific enzymes, alpha and beta amylases, amyloglucosidase, aligoglucosidase, invertase, maltase, cellulase, and hemi-cellulase are disclosed in U.S. Pat. Nos. 5,806,597 and 5,067,566, incorporated herein by reference.

A breaking agent or breaker may be used “as is” or be encapsulated and activated by a variety of mechanisms including crushing by formation closure or dissolution by formation fluids. Such techniques are disclosed, for example, in U.S. Pat. Nos. 4,506,734; 4,741,401; 5,110,486; and 3,163,219, incorporated herein by reference.

### Inorganic Acids

Suitable inorganic acids include, without limitation, any inorganic acid. Exemplary examples include, without limitation, hydrogen chloride, sulfuric acid, phosphoric acid, or mixtures thereof.

### Organic Acids

Suitable organic acids include, without limitation, any organic acid. Exemplary examples include, without limitation, formic acid, acetic acid, propionic acid, or mixtures thereof.

### Inorganic Bases

Suitable inorganic bases include, without limitation, any inorganic base. Exemplary examples include, without limitation, sodium hydroxide, sodium bicarbonate, sodium carbonate, sodium bicarbonate, potassium hydroxide, potassium bicarbonate, potassium carbonate, or mixtures thereof.

### Organic Bases

Suitable organic acids include, without limitation, any organic base. Exemplary examples include, without limitation, sodium tert-butoxide, potassium tert-butoxide, choline hydroxide, or mixtures thereof.

### Friction Reducing Agents

Suitable friction-reducing polymers for use in this disclosure include, without limitation, one or more anionic polymers, one or more cationic polymers, one or more amphoteric polymers, or any combination thereof. Exemplary examples include, without limitation, one or more acrylamide copolymers, one or more anionic acrylamide copolymers, one or more cationic acrylamide copolymers, one or more nonionic acrylamide copolymers, one or more amphoteric acrylamide copolymers, one or more polyacrylamides, one or more polyacrylamide derivatives, one or more polyacrylate, one or more polyacrylate derivative, one or more polymethacrylate, one or more polymethacrylate derivatives, and any mixture or combination thereof. Exemplary examples of suitable FR polymers include, without limitation, polyacrylates, polyacrylate derivatives, polyacrylate copolymers, polymethacrylates, polymethacrylate derivatives, polymethacrylate copolymers, polyacrylamide, polyacrylamide derivatives, polyacrylamide copolymers, acrylamide copolymers, polysaccharides, polysaccharide derivatives, polysaccharide copolymers, synthetic polymers, superabsorbent polymers, and any combination thereof. Exemplary examples of water soluble FR polymers include, without limitation, polymers containing one or more of the following monomers: acrylamide, acrylic acid, methacrylic acid, vinyl acetate, vinyl sulfonic acid, N-vinyl acetamide, N-vinyl formamide, itaconic acid, acrylic acid ester, methacrylic acid ester, ethoxylated-2-hydroxyethyl acrylate, ethoxylated-2-hydroxyethyl methacrylate, 2-hydroxyethyl acrylate, 2-hydroxyethylmethyl methacrylate, 2-hydroxypropyl acrylate, 2-hydroxypropyl methacrylate, hydroxymethyl styrene, 2-acrylamido-2-methylpropane sulfonic acid (AMPS), acrylamido tertiary butyl sulfonic acid (ATBS), 2-(meth)acrylamido-2-methylpropane sulfonic acid, 2-amino-2-methyl-1-propanol (AMP), N,N-dimethylacrylamide (DMAA), a salt of any of the foregoing, and any combination thereof. In certain embodiments, the FR polymers include one or more copolymers including acrylamide and AMPS. In other embodiments, the FR polymers may comprise high molecular weight, linear polymers. In certain embodiments, the one or more friction reducing polymers include one or more monomers. The one or more monomers include acrylamide, acrylic acid, 2-acrylamido-2-methylpropane sulfonic acid, acrylamido tertiary butyl sulfonic acid, a salt of any of the foregoing, and any mixture or combination thereof. In other embodiments, the water-based FR polymers have molecular weights ranging from about 100,000 to about 40,000,000, from about 200,000 to about 35,000,000, from about 300,000 to about 30,000,000, from about 400,000 to about 25,000,000, or from about 500,000 to about 20,000,000.

### Hydration Delaying Salt Compositions

Suitable hydration delaying salt compositions for use in this disclosure include, without limitation, ammonium sulfate or a mixture of ammonium sulfate and one or more other salts. The one or more other salts include, without limitation, one or more carbonate salts, one or more sulfate salts, one or more phosphate salts, one or more magnesium salts, one or more bromide salts, one or more formate salts, one or more acetate salts, one or more chloride salts, one or more fluoride salts, a bicarbonate salts, one or more nitrate salts, and any mixture or combination thereof. Exemplary examples of the one or more carbonate salts include, without limitation, ammonium carbonate, sodium carbonate, potassium carbonate, aluminum carbonate, magnesium carbonate, calcium carbonate, barium carbonate, strontium carbonate, zinc carbonate, other metal carbonates, or any mixture or combination thereof. Exemplary examples of the one or more phosphate salts include, without limitation, ammonium sulfate, sodium sulfate, potassium sulfate, aluminum sulfate, magnesium sulfate, calcium sulfate, barium sulfate, strontium sulfate, zinc sulfate, other metal sulfates, or any mixture or combination thereof. Exemplary examples of the one or more chloride salts include, without limitation, ammonium chloride, sodium chloride, potassium chloride, calcium chloride, magnesium chloride, strontium chloride, barium chloride, other metal chlorides, or any mixture or combination thereof. Exemplary examples of the one or more bromide salts include, without limitation, sodium bromide, potassium bromide, calcium bromide, magnesium bromide, zinc bromide, strontium bromide, other metal bromides, or any mixture or combination thereof. Exemplary examples of the one or more bicarbonates include, without limitation, sodium bicarbonate, potassium bicarbonate, other metal bicarbonates, or any mixture or combination thereof. Exemplary examples of the one or more nitrate salts include, without limitation, sodium nitrate, potassium nitrate, calcium nitrate, magnesium nitrate, zinc nitrate, strontium nitrate, other metal nitrate, or any mixture or combination thereof.

In other embodiments, the hydration delaying salt compositions may include, without limitation, divalent salts such as calcium and/or magnesium salts and monovalent salts such as ammonium and/or potassium salts work to prevent hydration as well.

In other embodiments, the hydration delaying salt compositions may include, without limitation, phosphate based salts such as potassium phosphate and/or variants such as potassium hexametaphosphate, which are capable of delaying or preventing FR polymer hydration.

In other embodiments, the hydration delaying salt compositions may include, without limitation, water-soluble potassium salts such as potassium citrate, potassium carbonate, etc., which are also capable of reducing or prevention FR polymer hydration. However, these salts are higher cost than hydration delaying salt compositions based on ammonium sulfate as the major component (greater than 50%).

In other embodiments, the hydration delaying salt compositions may include, without limitation, multivalent salts such as zinc chloride, aluminum chloride, iron chloride, zinc sulfate, aluminum sulfate, iron sulfate, or any combination thereof.

In other embodiments, the hydration delaying salt compositions may include, without limitation, double salt equivalents such as magnesium ammonium sulfates, calcium ammonium sulfates, aluminum ammonium sulfates, iron ammonium sulfates, nickel ammonium sulfates, copper ammonium sulfates, similar metal ammonium salts, or any combination thereof.

### Friction Reducing Composition Additives

### Suspending Agents

Suitable suspending agents for use in this disclosure include, without limitation, a bentonite clay, a phyllosilicate clay, or any combination thereof. In certain embodiments, the clay may include, without limitation, BYK OPTIBENT-987, a clay commercially available from BYK Additives and Instruments of Gonzales, Tex. In other embodiments, the clay may include nano-structures and/or micro-structures.

### Gel-Bridging Agents

Suitable gel-bridging agents for use in this disclosure include, without limitation, polyethylene glycols such as PEG 200, PEG 300, PEG 400, PEG 500, or similar polyethylene polymers, polypropylene glycols, polyethylene/propylene glycols, other polyalkylene oxide polymers, or any mixture thereof.

### pH Adjusting Agents

Suitable pH adjusting agents for use in this disclosure include, without limitation, organic acids such as fatty acid, diacids, polyacids, citric acid, oxalic acid, ascorbic acid, isocitric acid, aconitic acid, propane-1,2,3-tricarboxylic acid, or any mixture thereof.

### Fatty Acids

Suitable fatty acids for use in this disclosure include, without limitation, any saturated fatty acid or unsaturated fatty acids or mixtures or combinations thereof suitable for a human, mammal or animal consumption. Exemplary fatty acids include short chain free fatty acids (SCFFA), medium chain free fatty acids (MCFFA), long chain free fatty acids (LCFFA), very-long-chain free fatty acids (VLCFFA) and mixtures or combinations thereof. SCFFA include free fatty acids having a carbyl tail group having less than between 4 and less than 8 carbon atoms (C₄ to C₈). MCFFA include free fatty acids having a carbyl group having between 8 and less than 14 carbon atoms (C₈ to C₁₄). LCFFA include free fatty acids having a carbyl group having between 14 and 24 carbon atoms (C₁₄-C₂₄). VLCFFA include free fatty acids having a carbyl group having greater than 24 carbon atoms (>C₂₄). Exemplary unsaturated fatty acids include, without limitation, myristoleic acid [CH₃(CH₂)₃CH═CH(CH₂)₇COOH, cis-Δ⁹, C:D 14:1, n-5], palmitoleic acid [CH₃(CH₂)₅CH═CH(CH₂)₇COOH, cis-Δ⁹, C:D 16:1, n-7], sapienic acid [CH₃(CH₂)₈CH═CH(CH₂)₄COOH, cis-Δ⁶, C:D 16:1, n-10], oleic acid [CH₃(CH₂)₇CH═CH(CH₂)₇COOH, cis-Δ⁹, C:D 18:1, n-9], linoleic acid [CH₃(CH₂)₄CH═CHCH₂CH═CH(CH₂)₇COOH, cis, cis-Δ⁹,Δ¹², C:D 18:2, n-6], α-Linolenic acid [CH₃CH₂CH═CHCH₂CH═CHCH₂CH═CH(CH₂)₇COOH, cis, cis, cis-Δ⁹,Δ¹², Δ¹⁵, C:D 18:3, n-3], arachidonic acid [CH₃(CH₂)₄CH═CHCH₂CH═CHCH₂CH═CHCH₂CH═CH(CH₂)₃COOH, cis, cis, cis, cis-Δ⁵Δ⁸, Δ¹¹, Δ¹⁴, C:D 20:4, n-6], eicosapentaenoic acid [CH₃CH₂CH═CHCH₂CH═CHCH₂CH═CHCH₂CH═CHCH₂CH═CH(CH₂)₃COOH], cis,cis,cis,cis, cis-Δ⁵,Δ⁸,Δ¹¹,Δ¹⁴,Δ¹⁷, 20:5, n-3], erucic acid [CH₃(CH₂)₇CH═CH(CH₂)₁₁COOH, cis-413, C:D 22:1, n-9], docosahexaenoic acid [CH₃CH₂CH═CHCH₂CH═CHCH₂CH═CHCH₂CH═CH CH₂CH═CHCH₂CH═CH(CH₂)₂COOH, cis, cis, cis, cis, cis, cis-Δ⁴,Δ⁷,Δ¹⁰,Δ¹³,Δ¹⁶,Δ¹⁹, C:D 22:6, n-3], or mixtures and combinations thereof.

Exemplary saturated fatty acids include, without limitation, lauric acid [CH₃(CH₂)₁₀COOH, C:D 12:0], myristic acid [CH₃(CH₂)₁₂COOH, C:D 14:0], palmitic acid [CH₃(CH₂)₁₄COOH, C:D 16:0], stearic acid [CH₃(CH₂)₁₆COOH, C:D 18:0], arachidic acid [CH₃(CH₂)₁₈COOH, C:D 20:0], behenic acid [CH₃(CH₂)₂₀COOH, C:D 22:0], lignoceric acid [H₃(CH₂)₂₂COOH, C:D 24:0], cerotic acid [CH₃(CH₂)₂₄COOH, C:D 26:0], or mixture or combinations thereof.

Exemplary saturated fatty acids include, without limitation, butyric (C₄), valeric (C₅), caproic (C₆), enanthic (C₇), caprylic (C₈), pelargonic (C₉), capric (C₁₀), undecylic (C₁₁), lauric (C₁₂), tridecylic (C₁₃), myristic (C₁₄), pentadecylic (C₁₅), palmitic (C₁₆), margaric (C₁₇), stearic (C₁₈), nonadecylic (C₁₉), arachidic (C₂₀), heneicosylic (C₂₁), behenic (C₂₂), tricosylic (C₂₃), lignoceric (C₂₄), pentacosylic (C₂₅), cerotic (C₂₆), heptacosylic (C₂₇), montanic (C₂₈), nonacosylic (C₂₉), melissic (C₃₀), hentriacontylic (C₃₁), lacceroic (C₃₂), psyllic (C₃₃), geddic (C₃₄), ceroplastic (C₃₅), hexatriacontylic (C₃₆), heptatriacontylic acid (C₃₇), octatriacontylic acid (C₃₈), nonatriacontylic acid (C₃₉), tetracontylic acid (C₄₀), and mixtures or combinations thereof. Unsaturated fatty acids include, without limitation, n-3 unsaturated fatty acids such as α-linolenic acid, stearidonic acid, eicosapentaenoic acid, and docosahexaenoic acid, n-6 unsaturated fatty acids such as linoleic acid, γ-linolenic acid, dihomo-γ-linolenic acid, and arachidonic acid, n-9 unsaturated fatty acids oleic acid, claidic acid, cicosenoic acid, erucic acid, nervonic acid, mead acid and mixtures or combinations thereof.

Exemplary unsaturated fatty acids include, without limitation, (a) ω-3 unsaturated fatty acids such as octenoic (8:1), decenoic (10:1), decadienoic (10:2), lauroleic (12:1), laurolinoleic (12:2), myristovaccenic (14:1), myristolinoleic (14:2), myristolinolenic (14:3), palmitolinolenic (16:3), palmitidonic (16:4), α-linolenic (18:3), stearidonic (18:4), dihomo-α-linolenic (20:3), cicosatetraenoic (20:4), eicosapentaenoic (20:5), clupanodonic (22:5), docosahexaenoic (22:6), 9,12,15,18,21-tetracosapentaenoic (24:5), 6,9, 12, 15, 18,21-tetracosahexaenoic (24:6), and mixtures or combinations thereof; (b) ω-5 unsaturated such as myristoleic (14:1), palmitovaccenic (16:1), α-cleostearic (18:3), β-cleostearic (trans-18:3) punicic (18:3), 7,10,13-octadecatrienoic (18:3), 9,12,15-cicosatrienoic (20:3), β-cicosatetraenoic (20:4), and mixtures or combinations thereof; (c) ω-6 unsaturated such as 8-tetradecenoic (14:1), 12-octadecenoic (18:1), linoleic (18:2), linolelaidic (trans-18:2), γ-linolenic (18:3), calendic (18:3), pinolenic (18:3), dihomo-linoleic (20:2), dihomo-γ-linolenic (20:3), arachidonic (20:4), adrenic (22:4), osbond (22:5), and mixtures or combinations thereof; (d) ω-7 unsaturated such as palmitoleic (16:1), vaccenic (18:1), rumenic (18:2), paullinic (20:1), 7,10,13-eicosatrienoic (20:3), and mixtures or combinations thereof; (c) ω-9 Unsaturated such as oleic (18:1), claidic (trans-18:1), gondoic (20:1), erucic (22:1), nervonic (24:1), 8,11-eicosadienoic (20:2), mead (20:3), and mixtures or combinations thereof; (f) ω-10 Unsaturated such as Sapienic (16:1); (g) ω-11 unsaturated such as gadoleic (20:1); (h) ω-12 Unsaturated such as 4-Hexadecenoic (16:1) Petroselinic (18:1) 8-Eicosenoic (20:1), and mixtures or combinations thereof; and (i) mixtures or combinations thereof.

### Diacids

Exemplary examples of saturate diacids include, without limitation, ethanedioic acid (oxalic acid), propanedioic acid (malonic acid), butanedioic acid (succinic acid), pentanedioic acid (glutaric acid), hexanedioic acid (adipic acid), heptanedioic acid (pimelic acid), octanedioic acid (suberic acid, nonanedioic acid (azelaic acid), decanedioic acid (sebacic acid), undecanedioic acid, dodecanedioic acid, tridecanedioic acid (brassylic acid), hexadecanedioic acid (thapsic acid), heneicosa-1,21-dioic acid (japanic acid), docosanedioic acid (phellogenic acid), triacontanedioic acid (equisetolic acid), and mixtures or combinations thereof. Exemplary examples of unsaturated diacids include, without limitation, (Z)-butenedioic acid (maleic acid), (E)-butenedioic acid (fumaric acid), (Z and E)-pent-2-enedioic acid (glutaconic acid), 2-decenedioic acid, dodec-2-enedioic acid (traumatic acid), (2E,4E)-hexa-2,4-dienedioic acid (muconic acid), and mixtures or combinations thereof.

### Poly Acids

Suitable poly carboxylic acid compounds for use a pH depending release agents include, without limitation, any poly carboxylic acid compound. Exemplary examples of water immiscible poly acids include, without limitation, dicarboxylic acids having carbyl or carbenyl groups having between 8 and 50 carbon atoms and mixtures or combinations thereof. Polymer carboxylic acids or polymers including carboxylic acid groups, where the polymers are oil soluble or are oils, not miscible with water. Exemplary example of hydrophilic poly acids include, without limitation, polyacrylic acid, polymethacrylic acid, polylactic acid, polyglycol acid, mixtures and combinations thereof, copolymers thereof, CARBOPOLR reagents available from Lubrizol Corporation (a registered trademark of the Lubrizol Corporation), other carboxylic acid containing polymers, or mixtures or combinations thereof.

### Hydroxy Acids

Suitable hydroxy acids include, without limitation, 2-hydroxyoleic acid, 2-hydroxytetracosanoic acid (cerebronic acid), 2-hydroxy-15-tetracosenoic acid (hydroxynervonic acid), 2-hydroxy-9-cis-octadecenoic acid, 3-hydroxypalmitic acid methyl ester, 2-hydroxy palmitic acid, 10-hydroxy-2-decenoic acid, 12-hydroxy-9-octadecenoic acid (ricinoleic acid), 1,13-dihydroxy-tetracos-9t-enoic acid (axillarenic acid), 3,7-dihydroxy-docosanoic acid (byrsonic acid), 9,10-dihydroxyoctadecanoic acid, 9,14-dihydroxyoctadecanoic acid, 22-hydroxydocosanoic acid (phellonic acid), 2-oxo-5,8,12-trihydroxydodecanoic acid (phaseolic acid), 9,10,18-trihydroxyoctadecanoic acid (phloionolic acid), 7,14-dihydroxydocosa-4Z,8,10,12,16Z, 19Z-hexaenoic acid (Maresin 1), 5S,12R,18R-trihydroxy-6Z,8E,10E,14Z,16E-cicosapentaenoic acid (resolvin E1), resolvin D1, 10,17S-docosatriene, (neuroprotectin D1).

### Treating Fluid Composition Additives

Suitable additional additives include proppants, acids, diverting agents, fluid loss control additives, gas, nitrogen, carbon dioxide, surface modifying agents, tackifying agents, foamers, corrosion inhibitors, scale inhibitors, catalysts, clay control agents, biocides, antifoam agents, bridging agents, flocculants, H₂S scavengers, CO₂ scavengers, oxygen scavengers, lubricants, viscosifiers, breakers, weighting agents, relative permeability modifiers, resins, surfactants, wetting agents, coating enhancement agents, filter cake removal agents, antifreeze agents (e.g., ethylene glycol), and the like. A person skilled in the art, with the benefit of this disclosure, will recognize the types of additives that may be included in the fluids of the present disclosure for a particular application.

### Nonionic and Amphoteric Polymers

Suitable nonionic and amphoteric polymers used in the present composition preferably exhibit a molecular weight within the range of about 8 million to about 14 million or ranging from about 10 million to 15 million or ranging from about 10 million to about 12 million. Additional information on this mixture of friction-reducing polymers for high TDS systems is disclosed in copending U.S. patent application Ser. No. 15/786,769 the disclosure of which is hereby incorporated by reference.

Exemplary polymers (anionic, cationic, or amphoteric) may also be homopolymers, copolymers, terpolymers, or high order mixed monomer polymers synthesized from one or more anionic monomers, cationic monomers, and/or neutral monomers. For copolymer and high order mixed monomer polymers, the monomers used may have similar reactivities so that the resultant amphoteric polymeric material has a random distribution of monomers. The anionic monomers may be any anionic monomer such as acrylic acid, methacrylic acid, 2-acrylamide-2-methylpropane sulfonic acid, and/or maleic anhydride. The cationic monomer may be any cationic monomer such as dimethyl-diallyl ammonium chloride, dimethylamino-ethyl methacrylate, and/or allyltrimethyl ammonium chloride. The neutral monomer may be any neutral monomer such as butadiene, N-vinyl-2-pyrrolidone, methyl vinyl ether, methyl acrylate, maleic anhydride, styrene, vinyl acetate, acrylamide, methyl methacrylate, and/or acrylonitrile. Other exemplary polymers may be a terpolymer synthesized from acrylic acid (AA), dimethyl diallyl ammonium chloride (DMDAC) or diallyl dimethyl ammonium chloride (DADMAC), and acrylamide (AM). The ratio of monomers in the terpolymer can generally be any ratio. A presently preferred ratio is about 1:1:1. Other exemplary amphoteric polymeric materials include approximately 30% polymerized AA, 40% polymerized AM, and 10% polymerized DMDAC or DADMAC with approximately 20% free residual DMDAC or DADMAC which is not polymerized due to lower relative reactivity of the DMDAC or DADMAC monomer.

### DETAILED DESCRIPTION OF THE DRAWING

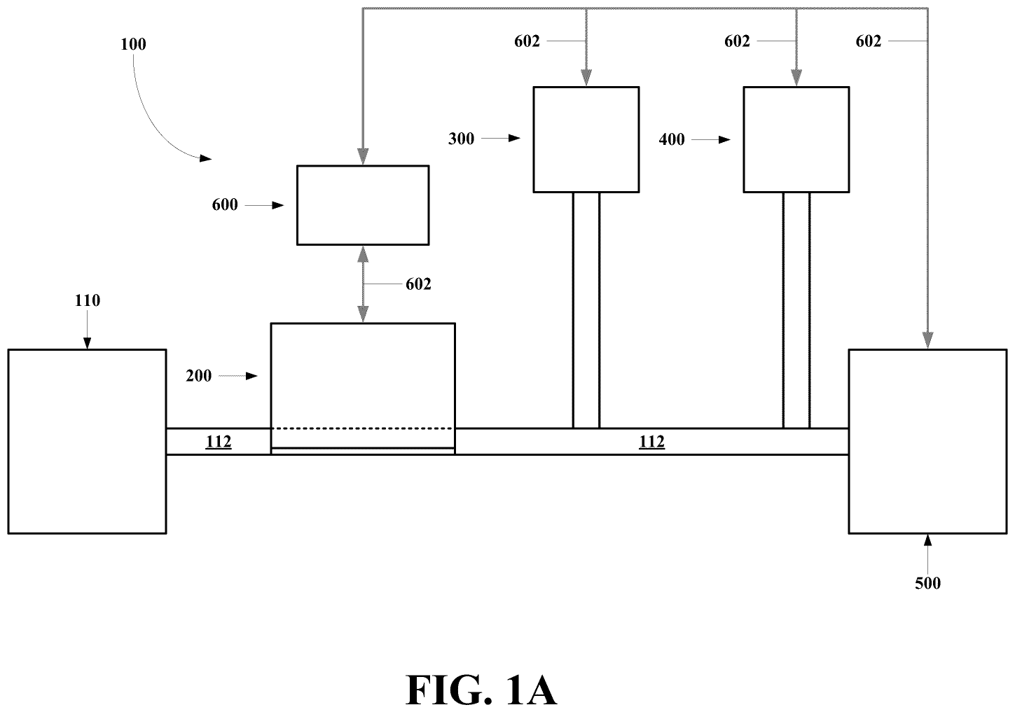

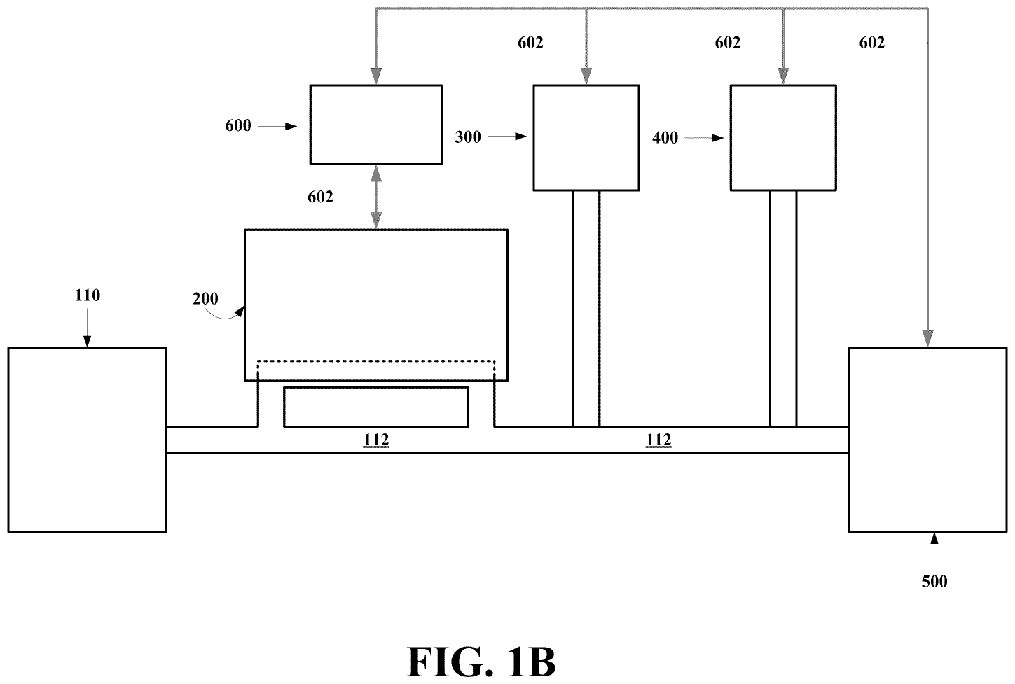

Referring now to FIG. 1A, an embodiment of a real-time or near real-time real-time, downhole fluid formulation apparatus/system, generally 100, is shown to include: (e) a water source assembly/subsystem 110 including a water supply conduit 112, (f) an in-line water sensor assembly/subsystem 200, (g) a downhole fluid component supply assembly/subsystem 300, (h) a friction reducing component supply assembly/subsystem 400, (i) a downhole fluid injection assembly/subsystem 500, (j) a control assembly/subsystem 600 including bi-directional communication pathways 602, and (k) a downhole fluid injection or circulation assembly/subsystem.

Referring now to FIG. 1B, another embodiment of a real-time or near real-time real-time, downhole fluid formulation apparatus/system, generally 100, is shown to include: (f) a water source assembly/subsystem 110 including a water supply conduit 112, (g) a stand-still, in-line water sensor assembly/subsystem 200, (h) a downhole fluid component supply assembly/subsystem 300, (i) a friction reducing component supply assembly/subsystem 400, (j) a downhole fluid injection assembly/subsystem 500, and (k) a control assembly/subsystem 600 including bi-directional communication pathways 602.

### In-Line Water Sensor Assembly/Subsystem

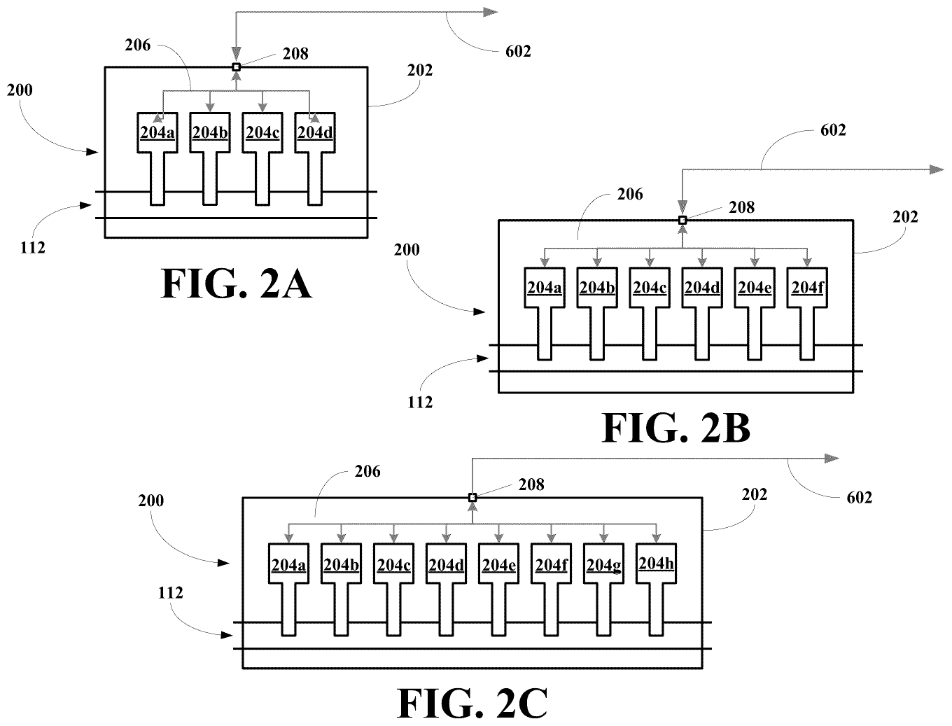

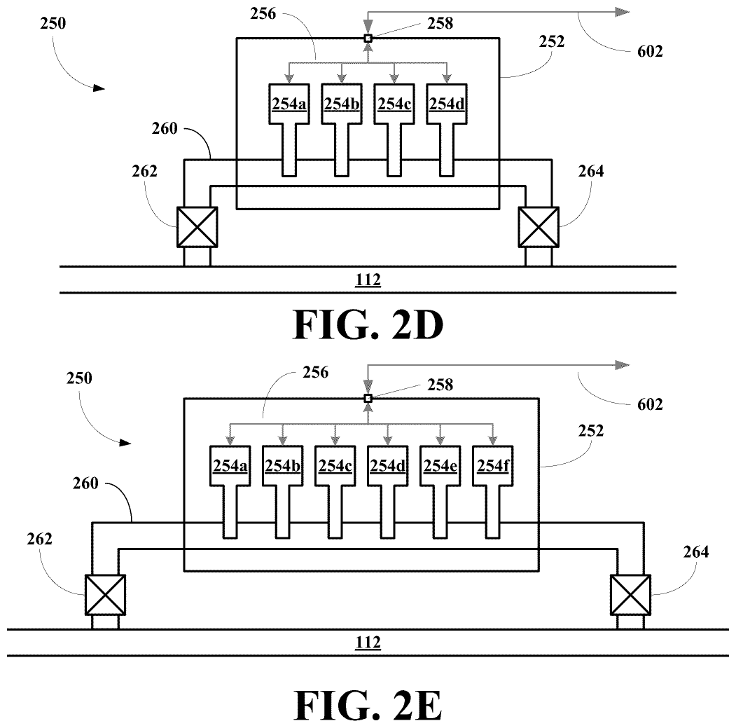

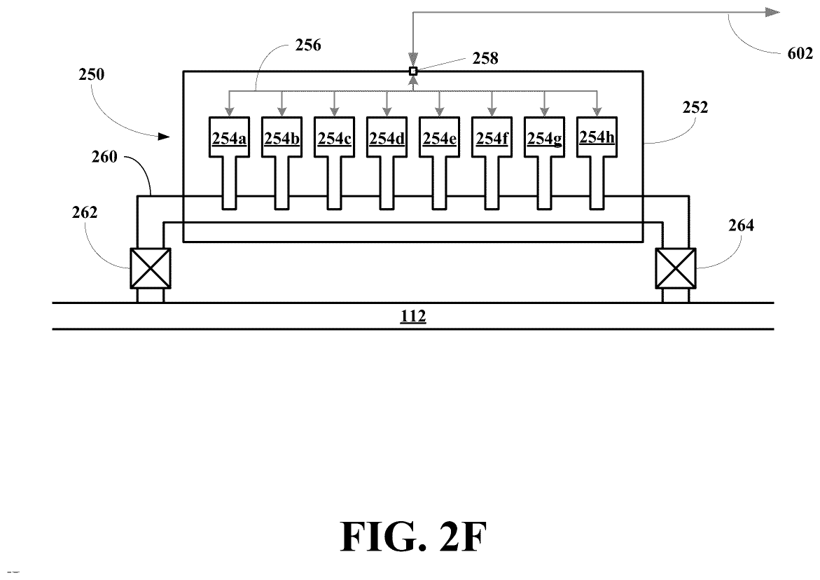

Referring now to FIG. 2A, the in-line water sensor assembly/subsystem 200 includes the in-line water sensor unit 202 including four chemical sensor 204a-d, wherein the sensing end of each of the sensor 204a-d are disposed in the water flowing through the conduit 112. The in-line water sensor assembly/subsystem 200 also includes data communication pathways 206 leading from the top of each of the sensor 204a-d to a communication fitting sensor 208. Each of the sensor 204a-d is designed to detect a different water property such salinity, conductivity, pH, or other water properties and/or a different ion concentration such as ion specific or chemical sensors that measure the concentration of specific ions or chemicals such as sodium ion, potassium, carbonate, hydronium, hydroxide, borates, carbon dioxide, oxygen, calcium, magnesium, titanium, zirconium, or other specific ions or chemicals.

Referring now to FIG. 2B, the in-line water sensor assembly/subsystem 200 includes the in-line water sensor unit 202 including four chemical sensor 204a-f, wherein the sensing end of each of the sensor 204a-f are disposed in the water flowing through the conduit 112. The in-line water sensor assembly/subsystem 200 also includes data communication pathways 206 leading from the top of each of the sensor 204a-f to a communication fitting sensor 208. Each of the sensor 204a-f is designed to detect a different water property such salinity, conductivity, pH, or other water properties and/or a different ion concentration such as ion specific or chemical sensors that measure the concentration of specific ions or chemicals such as sodium ion, potassium, carbonate, hydronium, hydroxide, borates, carbon dioxide, oxygen, calcium, magnesium, titanium, zirconium, or other specific ions or chemicals.

Referring now to FIG. 2C, the in-line water sensor assembly/subsystem 200 includes the in-line water sensor unit 202 including four chemical sensor 204a-h, wherein the sensing end of each of the sensor 204a-h are disposed in the water flowing through the conduit 112. The in-line water sensor assembly/subsystem 200 also includes data communication pathways 206 leading from the top of each of the sensor 204a-h to a communication fitting sensor 208. Each of the sensor 204a-h is designed to detect a different water property such salinity, conductivity, pH, or other water properties and/or a different ion concentration such as ion specific or chemical sensors that measure the concentration of specific ions or chemicals such as sodium ion, potassium, carbonate, hydronium, hydroxide, borates, carbon dioxide, oxygen, calcium, magnesium, titanium, zirconium, or other specific ions or chemicals.

Referring now to FIG. 2D, the in-line water sensor assembly/subsystem 250 includes the in-line water sensor unit 252 including four chemical sensor 254a-d, wherein the sensing end of each of the sensor 204a-d are disposed in the water flowing through the conduit 112. The in-line water sensor assembly/subsystem 200 also includes data communication pathways 256 leading from the top of each of the sensor 204a-d to a communication fitting sensor 258. The in-line water sensor assembly/subsystem 250 also includes a stand-still conduit 260 including a first control valve 262 and a second control valve 264. The control valves 262 and 264 are opened for a time sufficient for the water in the stand-still conduit 260 to be flushed to old water. Then, the control valves 262 and 264 are closed and then the sensors 266a-d. Each of the sensor 204a-d is designed to detect a different water property such salinity, conductivity, pH, or other water properties and/or a different ion concentration such as ion specific or chemical sensors that measure the concentration of specific ions or chemicals such as sodium ion, potassium, carbonate, hydronium, hydroxide, borates, carbon dioxide, oxygen, calcium, magnesium, titanium, zirconium, or other specific ions or chemicals.

Referring now to FIG. 2E, the in-line water sensor assembly/subsystem 250 includes the in-line water sensor unit 252 including four chemical sensors 254a-f, wherein the sensing end of each of the sensor 204a-f are disposed in the water flowing through the conduit 112. The in-line water sensor assembly/subsystem 250 also includes data communication pathways 256 leading from the top of each of the sensor 204a-f to a communication fitting sensor 258. The in-line water sensor assembly/subsystem 250 also includes a stand-still conduit 260 including a first control valve 262 and a second control valve 264. The control valves 262 and 264 are opened for a time sufficient for the water in the stand-still conduit 260 to be flushed to old water. Then, the control valves 262 and 264 are closed and then the sensors 266a-f. Each of the sensor 204a-f is designed to detect a different water property such salinity, conductivity, pH, or other water properties and/or a different ion concentration such as ion specific or chemical sensors that measure the concentration of specific ions or chemicals such as sodium ion, potassium, carbonate, hydronium, hydroxide, borates, carbon dioxide, oxygen, calcium, magnesium, titanium, zirconium, or other specific ions or chemicals.

Referring now to FIG. 2F, the in-line water sensor assembly/subsystem 250 includes the in-line water sensor unit 252 including four chemical sensor 254a-h, wherein the sensing end of each of the sensor 204a-h are disposed in the water flowing through the conduit 112. The in-line water sensor assembly/subsystem 200 also includes data communication pathways 256 leading from the top of each of the sensor 204a-h to a communication fitting sensor 258. The in-line water sensor assembly/subsystem 250 also includes a stand-still conduit 260 including a first control valve 262 and a second control valve 264. The control valves 262 and 264 are opened for a time sufficient for the water in the stand-still conduit 260 to be flushed to old water. Then, the control valves 262 and 264 are closed and then the sensors 266a-f. Each of the sensor 204a-h is designed to detect a different water property such salinity, conductivity, pH, or other water properties and/or a different ion concentration such as ion specific or chemical sensors that measure the concentration of specific ions or chemicals such as sodium ion, potassium, carbonate, hydronium, hydroxide, borates, carbon dioxide, oxygen, calcium, magnesium, titanium, zirconium, or other specific ions or chemicals.

### Downhole Fluid Component Supply Assembly/Subsystem

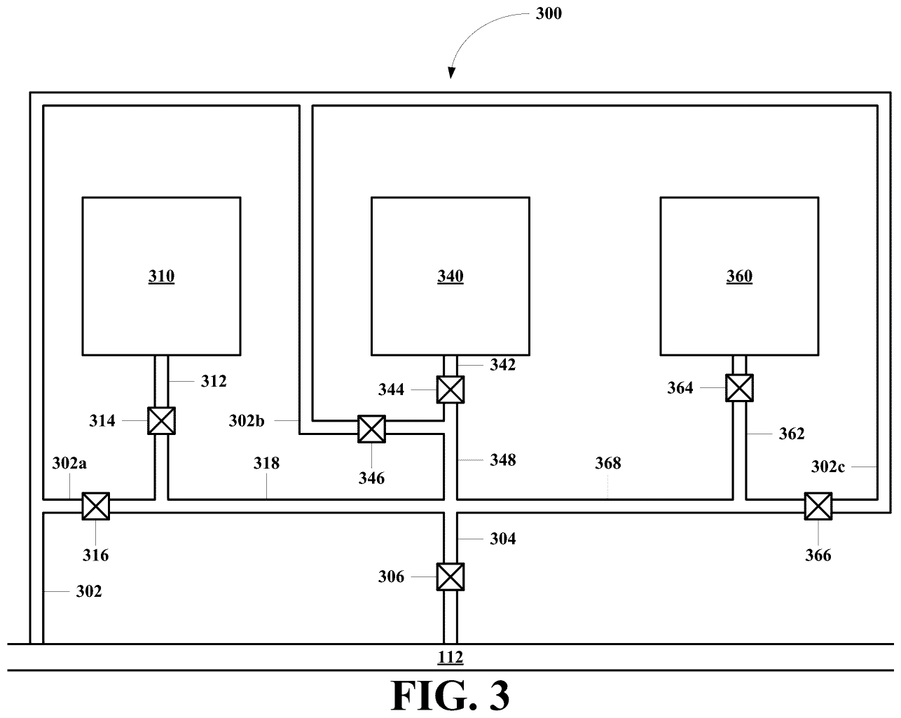

Referring now to FIG. 3, an embodiment of a downhole fluid component supply assembly/subsystem, generally 300, is shown to include a water conduit 302, an optimized first downhole fluid outlet conduit 304 having an optimized first downhole fluid control valve 306

The downhole fluid component supply assembly/subsystem 300 also includes a polymer reservoir 310, a polymer supply conduit 312 having a polymer composition control valve 314, a water conduit 302a having a first water control valve 316, and a polymer fluid outlet conduit 318.

The downhole fluid component supply assembly/subsystem 300 also includes a cross-link composition reservoir 340, a cross-link composition supply conduit 342 having a cross-link composition control valve 344, a second water conduit 302b having a second water control valve 346, and a cross-link fluid outlet conduit 348.

The downhole fluid component supply assembly/subsystem 300 also includes a downhole fluid additive composition reservoir 360, a downhole fluid additive composition supply conduit 362 having a downhole fluid additive control valve 364, a third water conduit 302c having a third water control valve 366, and a downhole additive fluid outlet conduit 368.

### Friction-Reducing Component Supply Assembly/Subsystem

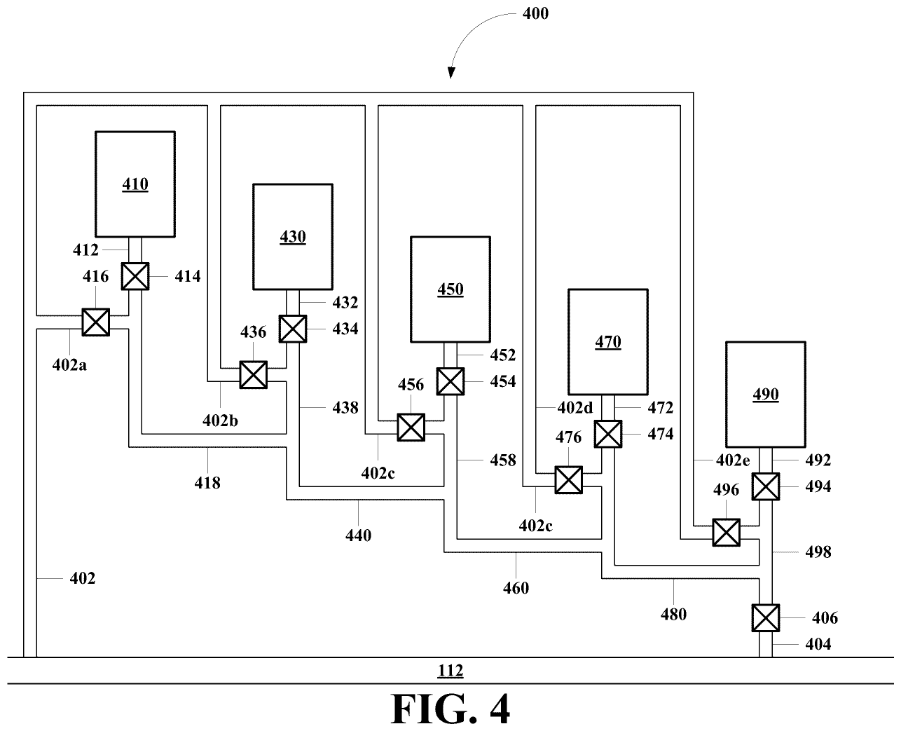

Referring now to FIG. 4, an embodiment of a friction-reducing (FR) component supply assembly/subsystem, generally 400, is shown to include a water conduit 402, an optimized downhole fluid outlet conduit 404 having an optimized FR downhole fluid control valve 406.

The friction-reducing component supply assembly/subsystem 400 also includes a first FR polymer reservoir 410, a first FR polymer supply conduit 412 having a first FR polymer control valve 414, a first water conduit 402a having a first water control valve 416, and a first FR polymer fluid outlet conduit 418.

The friction-reducing component supply assembly/subsystem 400 also includes a second FR polymer composition reservoir 430, a second FR composition supply conduit 432 having a second FR polymer control valve 434, a second water conduit 402b having a second water control valve 436, a second FR polymer fluid outlet conduit 438, and a first combined FR polymer fluid conduit 440. If the second FR polymer fluid and the first FR polymer fluid are to form part of the optimized FR polymer fluid, then the two FR polymer fluids are combined and flow through the combined FR polymer fluid conduit 440.

The friction-reducing component supply assembly/subsystem 400 also includes a third FR polymer composition reservoir 450, a third FR composition supply conduit 452 having a third FR polymer control valve 454, a third water conduit 402c having a third water control valve 456, and a third FR polymer fluid outlet conduit 458.

The friction-reducing component supply assembly/subsystem 400 also includes a fourth FR polymer composition reservoir 470, a fourth FR polymer composition supply conduit 472 having a fourth FR polymer control valve 474, a fourth water conduit 402c having a fourth water control valve 476, and a fourth FR polymer fluid outlet conduit 478.

The friction-reducing component supply assembly/subsystem 300 also includes a FR additive composition reservoir 490, a FR additive composition supply conduit 492 having a FR additive control valve 494, a fifth water conduit 302c having a fifth water control valve 496, and a FR additive fluid outlet conduit 498.

### Downhole Fluid Injection Assembly/Subsystem

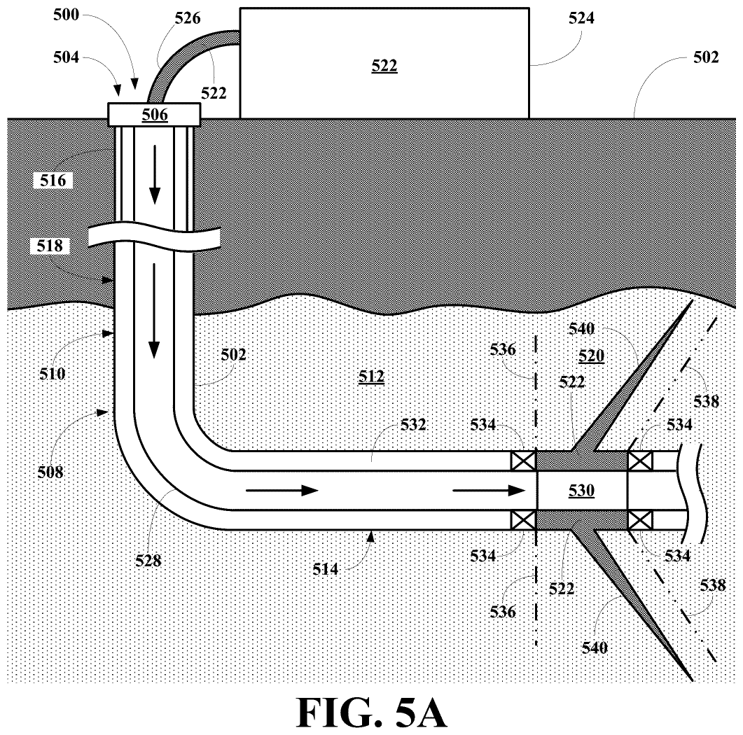

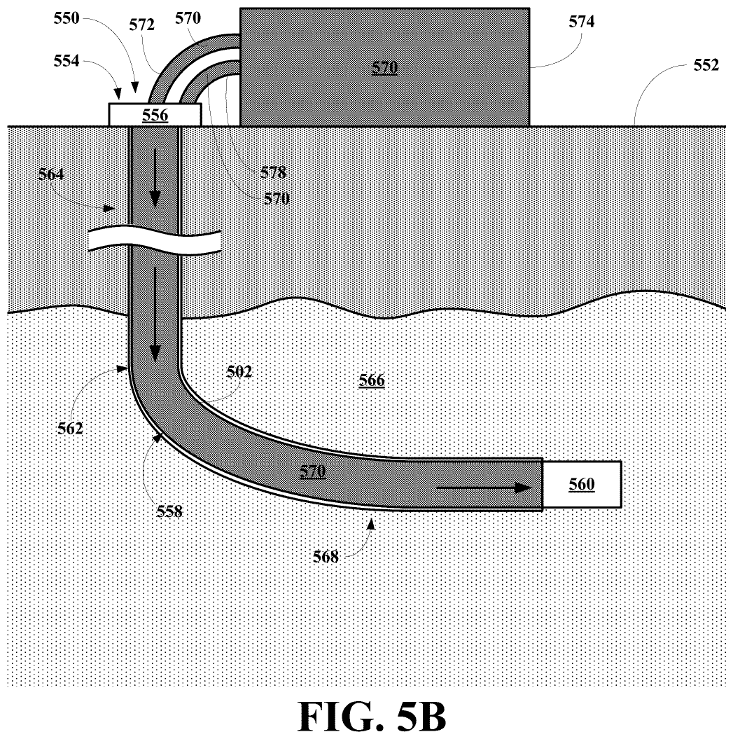

Referring now to FIG. 5A, an embodiment of an optimized treating downhole fluid wellsite injection assembly/subsystem, generally 500, located on a surface 502 at a location 504. The downhole fluid injection assembly/subsystem 500 includes a well head assembly 506 and a wellbore or borehole 508. The borehole 508 extends vertically from the surface 502 via a vertical section 510 thereof. Once the borehole 508 penetrates an oil and/or gas bearing subterranean formation 512, the borehole 508 extends horizontally into the formation 512 via a horizontal portion 514. Of course, it should be recognized that the borehole 508 may include horizontal, vertical, slant, curved, and other types of borehole geometries and orientations, and the fracturing treatment may be applied to an oil and/or gas subterranean zone surrounding any portion of the wellbore.

The borehole 508 also includes a casing 516 that is generally cemented in place or otherwise secured to a borehole wall 518. Of course, the borehole 508 may be uncased or include uncased sections. Perforations may also be formed in the casing 516 to allow fracturing fluids and/or other materials to flow into a portion 520 of the formation 512. In cased wells, perforations may be formed using shape charges, a perforating gun, hydro-jetting and/or other tools into the portion 520.

During a treating operation, the portion 520 of the formation 512 surrounding the borehole 508 will be exposed to a treating fluid 522. The treating fluid 522 will be supplied to the borehole 508 from a treating fluid reservoir 524 via a treating fluid supply line 526. In the present configuration, the fracturing fluid 522 is forwarded to the portion 520 via a work string 528 extending from the well head assembly 506 into the borehole 508. The treating fluid supply system 524 is coupled to or associated with the work string 528 including an end assembly 530 to pump the treating fluid 522 into and through the working string 528 under treating conditions of pressure, flow rate, etc., out of the end 530, and into the portion 520. The working string 528 may include coiled tubing, jointed pipe, and/or other structures that allow fluid to flow into the borehole 508. The working string 528 may also include flow control devices, bypass valves, ports, and/or other tools or well devices that control a flow of treating fluid from the interior of the working string 528 into the portion or zone 520 of the formation 512. For example, the working string 528 may also include ports adjacent the wellbore wall 518 to directly communicate the treating fluid 522 into the portion 520 of the formation 512, and/or the working string 528 may include ports that are spaced apart (several feet to hundreds of feet apart) from the borehole wall 518 to communicate the treating fluid 522 into an annulus 532 in the wellbore between the working string 528 and the borehole wall 518.

The working string 528 and/or the borehole 508 may include one or more sets of packers 534 that seal the annulus between the working string 528 and borehole 508 to isolate the portion 520 into which the treating fluid 522 will be pumped or injected. Two of the packers 534 are disposed uphole to define an uphole boundary 536 of the portion 520 and two of the packers 534 are disposed downhole to define an end 538 of the portion 520. When the treating fluid 522 is introduced into working string 528 a sufficient hydraulic pressure, one or more fractures 540 may be created in the portion 520. If the treating fluid is fracturing fluid including proppant, then the pressure will force the proppant-containing fracturing fluid into the portion 520 creating fractures 540. Depending on the pressure and pumping sequence, the proppant-containing fracturing fluid proppant will remain in the fractures 540 in a desired format to “prop” open the fractures 540 after pressure is removed so that fluids may flow more freely through the fractures 540 from the portion 520 into the borehole 508 and to the well head 506 for removal. In certain embodiments, multiple portions, zones, or intervals in the same formation 512 may be successively isolated and treated in a similar manner.

### Downhole Fluid Circulation Assembly/Subsystem

Referring now to FIG. 5B, an embodiment of an optimized drilling fluid circulation assembly/subsystem, generally 550, located on a surface 552 at a location 554. The drilling fluid circulation assembly/subsystem 550 includes a well head assembly 556, a drill string 558 ending in a drilling unit 560 to form a wellbore or borehole 562. The borehole 558 extends vertically from the surface 552 via a vertical section 564 thereof. Once the borehole 558 penetrates an oil and/or gas bearing subterranean formation 566, the borehole 558 then extends horizontally into the formation 566 via a horizontal portion 568. Of course, it should be recognized that the drilling string 558 and the drilling unit 560 may form a borehole 558 including horizontal, vertical, slant, curved, and other types of borehole geometries and orientations.

During a drilling operation, a drilling fluid 570 is circulated through the drill string 558 and the drilling unit 560. The drilling fluid 570 will be supplied to the drilling string 558 and the drilling unit 560 via a drilling fluid supply conduit 572 coming from a drilling fluid supply unit 574. In the present configuration, the drilling fluid 570 is circulated through the drill string 558 and the drilling unit 560 and returns through an annular space 576 between the drill string 558 and the borehole 562 and back to the forwarded to the drilling fluid supply unit 574 via a return conduit 578. Of course, the drill fluid is prepared via the system 100 and may be modified while drilling.

### Control Assembly/Subsystem

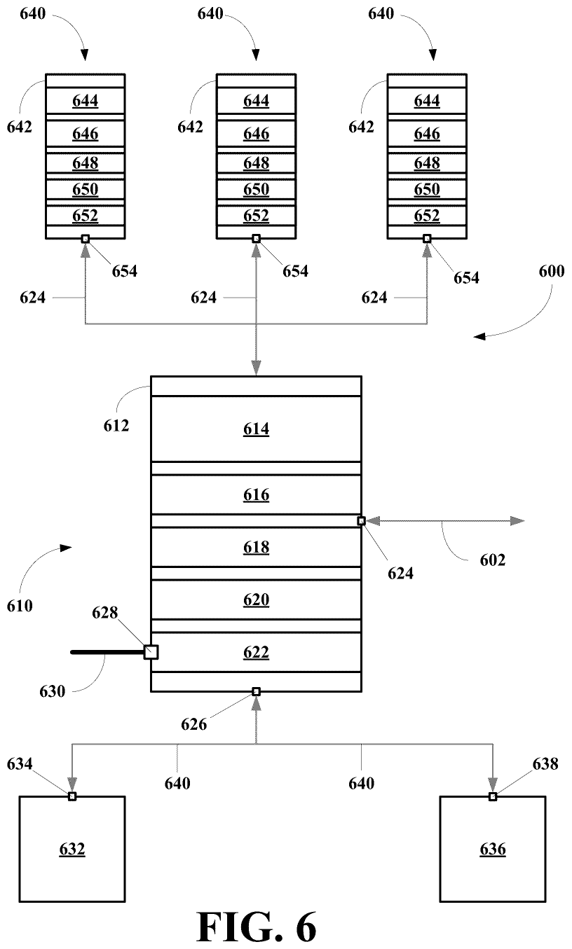

Referring now to FIG. 6, an embodiment of a control assembly/subsystem, generally 600, is shown to include a control unit 610 having a housing 612. The housing 612 including a processing unit 614, a memory 616, hardware and software communication unit 618, a mass storage unit 620, a power supply unit 622, a first communication port 624, a second communication port 626, a power supply port 628, and a power cable 630. The control unit 610 also includes an output unit 632 such as a display device having a communication port 634 and an input unit 636 such as a keyboard, mouse, or the like having a communication port 638. The output unit 632 and the input unit 636 are in bi-directional communication with the processing unit 614 via bi-directional pathways 640 through the communication port 626 to the communication ports 634 and 638. It should be recognized that the output unit 634 and the input unit 626 may be a single device such as a touch screen. It should also be recognized that the communication pathways 640 may be wired or wireless.

The control assembly/subsystem 600 may also include remote control units 642. Each of the remote control units 640 includes a display unit 644 (such as a touch screen), a processing unit 646, a memory 648, hardware and software communication unit 650, a mass storage unit 652, and a power supply unit 654.

### Method Flow Diagrams

### Treating Fluids and Formation Injection

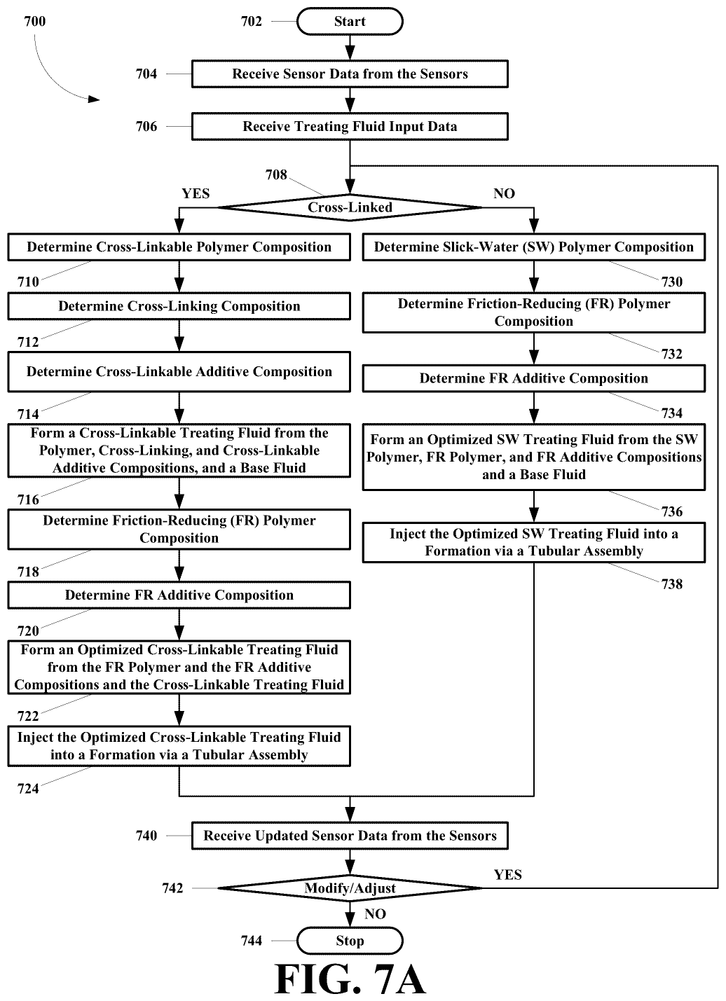

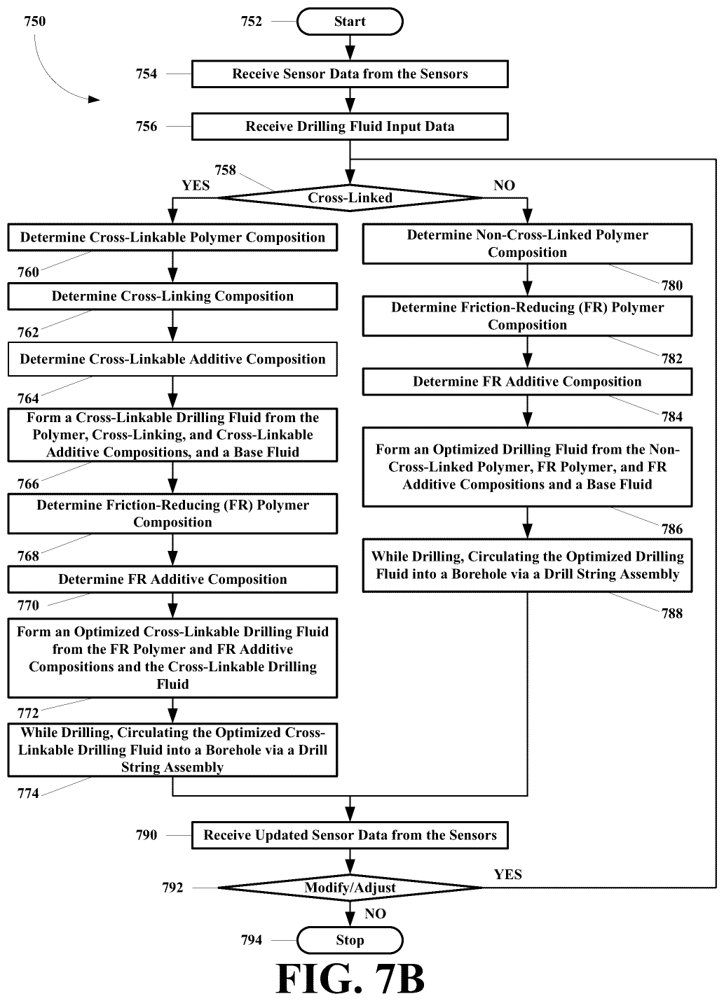

Referring now to FIG. 7A, an embodiment of a flow diagram of a method of treating a formation, generally 700, is shown to include a start step 702. Once started, control proceeds to a receive sensor data step 704, wherein sensor data is received. The sensor data includes data transmitted from all of the sensors in the sensor assembly/subsystem. Control then proceeds to a receive treating fluid input data step 706, wherein the input data is entered by an operator or received from a treating simulation program and includes a type of treating fluid to be used, the properties of the treating fluid to be used, a type of base fluid to be used, and the properties of the base fluid to be used. Control then proceeds to a cross-linked test step 708, wherein the treating fluid input data is used to determine if the treating fluid is a cross-linkable treating fluid or a non-cross-linkable or slick-water treating fluid.

### Cross-Linked or YES Path

If the treating fluid is a cross-linkable treating fluid, then control proceeds along a YES path to a determine a cross-linkable polymer composition step 710, wherein a type and an amount of one or more cross-linkable polymers to be used in the cross-linkable polymer composition are determined based on the sensor data and the treating fluid input data.

Control then proceeds to a determine a cross-linking composition step 712, wherein a type and an amount of one or more cross-linking agents to be used in the cross-linking composition are determined based on the sensor data and the treating fluid input data.

Control then proceeds to a determine a cross-linkable additive composition step 714, wherein a type and an amount of one or more cross-linkable additive to be used in the cross-linkable additive composition are determined based on the sensor data and the treating fluid input data.

Control then proceeds to form a cross-linkable treating fluid step 716, wherein the cross-linkable treating fluid is formed from the cross-linkable polymer composition, the cross-linking composition, the cross-linkable additive composition, and a base fluid.

Control then proceeds to a determine a friction-reducing (FR) polymer composition step 718, wherein a type and an amount of one or more FR polymers to be used in the FR polymer composition are determined based on the sensor data and the treating fluid input data.

Control then proceeds to a determine a FR additive composition step 720, wherein a type and an amount of one or more FR additive composition to be used in the FR additive composition are determined based on the sensor data and the treating fluid input data.

Control then proceeds to a form an optimized treating fluid step 722, wherein the optimized treating fluid is formed from the cross-linkable treating fluid, the FR polymer composition, and the FR additive composition. It should be recognized that the composition may be mixed with additional base fluid, if the polymers are dry polymers or the composition needs to be diluted.

Control then proceeds to an inject the optimized treating fluid step 724, wherein the optimized treating fluid is injected into a formation through a tubular assembly under treating conditions.

### Non-Cross-Linked or NO Path

If the treating fluid is a non-cross-linked or a slick-water (SW) treating fluid, then control proceeds from the cross-linked test step 708 along a NO path to a determine a SW polymer composition step 730, wherein a type and an amount of one or more SW polymers to be used in the SW polymer composition are determined based on the sensor data and treating fluid input data.

Control then proceeds to a determine a friction-reducing (FR) polymer composition step 732, wherein a type and an amount of one or more FR polymers to be used in the FR polymer composition are determined based on the sensor data and the treating fluid input data.

Control then proceeds to a determine a FR additive composition step 734, wherein a type and an amount of one or more FR additives to be used in the FR additive composition are determined based on the sensor data and the treating fluid input data.

Control then proceeds to a form an optimized downhole treating fluid step 736, wherein the optimized downhole treating fluid is formed the SW polymer composition, the FR polymer composition, the FR additive composition, and a base fluid.

Control then proceeds to an inject the optimized SW treating fluid step 738, wherein the optimized SW treating fluid is injected into a formation through a tubular assembly under treating conditions. It should be recognized that the composition may be mixed with additional base fluid, if the polymers are dry polymers or the composition needs to be diluted.

Control then proceeds from the inject step 724 or the inject step 738 to a receive updated sensor data step 740, wherein updated sensor data is received during the treating operation.

Control then proceeds from the receive updated sensor data step 740 to a modify or adjust a composition of the optimized cross-linkable treating fluid or the optimized SW treating fluid step 742.

If the updated sensor data indicate that the optimized cross-linkable treating fluid or the optimized SW treating fluid needs to be modified or adjusted during the treating operation, then control proceeds along a YES path back to the cross-linked test step 708, wherein the process of modifying or adjusting the treating fluid proceeds as set forth above for the cross-linked YES path and the non-cross-linked NO path.

If the sensor data indicates that the treating fluid does not need to be modified, then control proceeds along a NO path to a stop step 744, wherein the stop step coincides with the cessation of the treating operation.

### Drilling Fluid and Drilling Fluid Circulation

Referring now to FIG. 7B, an embodiment of a flow diagram of a method of drilling a borehole into a formation, generally 750, is shown to include a start step 752. Once started, control proceeds to a receive sensor data step 754. The sensor data includes data transmitted from all of the sensors in the sensor assembly/subsystem. Control then proceeds to a receive drilling fluid input data step 756, wherein the input data is entered by an operator or received from a treating simulation program and includes a type of treating fluid to be used, the properties of the treating fluid to be used, a type of base fluid to be used, and the properties of the base fluid to be used. Control then proceeds to a cross-linked test step 758, wherein the drilling fluid input data is used to determine if the drilling fluid is a cross-linkable drilling fluid or a non-cross-linkable drilling fluid.

### Cross-Linked or YES Path

If the drilling fluid is a cross-linked drilling fluid, then control proceeds along a YES path to a determine a cross-linkable polymer composition step 760, wherein a type and an amount of one or more cross-linkable polymers to be used in the cross-linkable polymer composition are determined based on the sensor data and the drilling fluid input data.

Control then proceeds to a determine a cross-linking composition step 762, wherein a type and an amount of one or more cross-linking agents to be used in the cross-linking composition are determined based on the sensor data and the drilling fluid input data.

Control then proceeds to a determine a cross-linkable additive composition step 764, wherein a type and an amount of one or more cross-linked additive to be used in the cross-linkable additive composition are determined based on the sensor data and the drilling fluid input data.

Control then proceeds to form a drilling fluid step 766, wherein the drilling fluid is formed from the cross-linkable polymer composition, the cross-linking composition, the cross-linkable additive composition, and a base fluid.

Control then proceeds to a determine a friction-reducing (FR) polymer composition step 768, wherein a type and an amount of one or more FR polymers to be used in the FR polymer composition are determined based on the sensor data and the drilling fluid input data.

Control then proceeds to a determine a FR additive composition step 770, wherein a type and an amount of one or more FR additives to be used in the FR additive composition are determined based on the sensor data and the drilling fluid input data.

Control then proceeds to a form an optimized drilling fluid step 772, wherein the optimized drilling fluid is formed from the cross-linkable drilling fluid, the FR polymer composition, and the FR additive composition. It should be recognized that the composition may be mixed with additional base fluid, if the polymers are dry polymers or the composition needs to be diluted.

Control then proceeds to a circulate an optimized drilling fluid step 774, wherein the optimized drilling fluid is circulated through a drilling string while drilling under drilling conditions.

### Non-Cross-Linked or NO Path

If the drilling fluid is to be a non-cross-linked drilling fluid, then control proceeds from the cross-linked test step 758 along a NO path to a determine a non-cross-linked polymer composition step 780, wherein a type and an amount of one or more non-cross-linked polymers to be used in the non-cross-linked polymer composition are determined based on the sensor data and the drilling fluid input data.

Control then proceeds to a determine a friction-reducing (FR) polymer composition step 782, wherein a type and an amount of one or more FR polymers to be used in the FR polymer composition are determined based on the sensor data and the drilling fluid input data.

Control then proceeds to a determine a FR additive composition step 784, wherein a type and an amount of one or more FR additives to be used in the FR additive composition are determined based on the sensor data and the drilling fluid input data.

Control then proceeds to a form an optimized non-cross-linked drilling fluid step 786, wherein the optimized non-cross-linked drilling fluid is formed from the non-cross-linked polymer composition, the FR polymer composition, the FR additive composition, and a base fluid.

Control then proceeds to a circulate the optimized non-cross-linked drilling fluid step 788, wherein the optimized non-cross-linked drilling fluid is circulated through a drilling string while drilling under drilling conditions.

Control then proceeds from the circulate step 774 and the circulate step 788 to a receive updated sensor data step 790, wherein the updated sensor data is received during the drilling operation.

Control then proceeds from the receive updated sensor data step 790 to a modify or adjust a composition of the optimized cross-linkable drilling fluid or the optimized non-cross-linkable drilling fluid step 792.

If the updated sensor data indicate that the optimized cross-linkable drilling fluid or the optimized non-cross-linkable drilling fluid needs to be modified or adjusted during the drilling operation, then control proceeds along a YES path back to the cross-linked test step 758, wherein the process of modifying or adjusting the optimized drilling fluid proceeds as set forth above for the cross-linked YES path and the non-cross-linked NO path.

If the sensor data indicates that the treating fluid does not need to be modified, then control proceeds along a NO path to a stop step 794, wherein the stop step coincides with the cessation of the drilling operation.

### Embodiments of the Disclosure

Embodiment 1. An apparatus comprising:
an aqueous base fluid supply assembly,
a stand-still in-line aqueous fluid sensor assembly,
a downhole sensor assembly,
a downhole fluid component supply assembly,
a friction-reducing component supply assembly,
a downhole fluid injection assembly, and
a control assembly,
wherein the apparatus is configured to adjust in real-time or near real-time, a composition of the downhole fluid based on data received from the in-line sensor assembly.

Embodiment 2. An apparatus comprising:
an aqueous base fluid supply assembly,
a stand-still in-line aqueous fluid sensor assembly,
a downhole sensor assembly,
a downhole fluid component supply assembly,
a friction-reducing component supply assembly,
a downhole circulation assembly, and
a control assembly,
wherein the apparatus is configured to adjust in real-time or near real-time, a composition of the downhole fluid based on data received from the in-line sensor assembly.

Embodiment 3. An apparatus comprising:
an aqueous base fluid supply assembly,
a flowing in-line aqueous fluid sensor assembly,
a downhole sensor assembly,
a downhole fluid component supply assembly,
a friction-reducing component supply assembly,
a downhole fluid injection assembly, and
a control assembly,
wherein the apparatus is configured to adjust in real-time or near real-time, a composition of the downhole fluid based on data received from the in-line sensor assembly.

Embodiment 4. An apparatus comprising:
an aqueous base fluid supply assembly,
a flowing in-line aqueous fluid sensor assembly,
a downhole sensor assembly,
a downhole fluid component supply assembly,
a friction-reducing component supply assembly,
a downhole circulation assembly, and
a control assembly,
wherein the apparatus is configured to adjust in real-time or near real-time, a composition of the downhole fluid based on data received from the in-line sensor assembly.

Embodiment 5. The Embodiment 1, wherein the stand-still or flowing in-line aqueous fluid sensor assembly is configured to generate real-time or near real-time data concerning a composition and properties of the aqueous base fluid and downhole sensor assembly is configured to generate real-time or near real-time data concerning a composition and properties of the downhole fluid and real-time or near real-time data concerning the treating or drilling properties.

Embodiment 6. The Embodiments 1 through 5, wherein the downhole fluid component supply assembly is configured to supply downhole fluid components to the aqueous base fluid based on the data to form a downhole fluid and the friction-reducing component supply assembly supplies friction-reducing components to the downhole fluid based on the data to form an optimized downhole fluid having a reduced or minimized percent drag reduction (% DR) value. In certain embodiments, the downhole sensor assembly may also include a program configured to generate modeling data for adjusting a composition and properties of the optimized downhole fluid based on the sensor and modeling data.

Embodiment 7. The Embodiments 1 through 6, wherein the control assembly is configured to utilize the data to adjust amount of the downhole fluid components, the friction-reducing components being added to the aqueous base fluid in real-time or near real-time.

Embodiment 8. A system comprising:
an aqueous base fluid supply subsystem,
a stand-still in-line aqueous fluid sensor subsystem,
a downhole sensor subsystem,
a downhole fluid component supply subsystem,
a friction-reducing component supply subsystem,
a downhole fluid injection subsystem, and
a control subsystem,
wherein the system is configured to adjust in real-time or near real-time, a composition of the downhole fluid based on data received from the in-line sensor assembly.

Embodiment 9. A system comprising:
an aqueous base fluid supply subsystem,
a stand-still in-line aqueous fluid sensor subsystem,
a downhole sensor subsystem,
a downhole fluid component supply subsystem,
a friction-reducing component supply subsystem,
a downhole circulation subsystem, and
a control subsystem,
wherein the system is configured to adjust in real-time or near real-time, a composition of the downhole fluid based on data received from the in-line sensor assembly.

Embodiment 10. A system comprising:
an aqueous base fluid supply subsystem,
a flowing in-line aqueous fluid sensor subsystem,
a downhole sensor subsystem,
a downhole fluid component supply subsystem,
a friction-reducing component supply subsystem,
a downhole fluid injection subsystem, and
a control subsystem,
wherein the system is configured to adjust in real-time or near real-time, a composition of the downhole fluid based on data received from the in-line sensor assembly.

Embodiment 11. A system comprising:
an aqueous base fluid supply subsystem,
a flowing in-line aqueous fluid sensor subsystem,
a downhole sensor subsystem,
a downhole fluid component supply subsystem,
a friction-reducing component supply subsystem,
a downhole circulation subsystem, and
a control subsystem,
wherein the system is configured to adjust in real-time or near real-time, a composition of the downhole fluid based on data received from the in-line sensor assembly.

Embodiment 12. The Embodiment 8, wherein the stand-still or flowing in-line aqueous fluid sensor subsystem is configured to generate real-time or near real-time data concerning a composition and properties of the aqueous base fluid and downhole sensor subsystem is configured to generate real-time or near real-time data concerning a composition and properties of the downhole fluid and real-time or near real-time data concerning the treating or drilling properties.

Embodiment 13. The Embodiments 8 through 12, wherein the downhole fluid component supply subsystem is configured to supply downhole fluid components to the aqueous base fluid based on the data to form a downhole fluid and the friction-reducing component supply subsystem supplies friction-reducing components to the downhole fluid based on the data to form an optimized downhole fluid having a reduced or minimized percent drag reduction (% DR) value. In certain embodiments, the downhole sensor subsystem may also include a program configured to generate modeling data for adjusting a composition and properties of the optimized downhole fluid based on the sensor and modeling data.

Embodiment 14. The Embodiments 8 through 13, wherein the control subsystem is configured to utilize the data to adjust amount of the downhole fluid components, the friction-reducing components being added to the aqueous base fluid in real-time or near real-time.

Embodiment 15. A method comprising:
supplying an aqueous base fluid from an aqueous based fluid supply assembly/subsystem,
passing the aqueous base fluid through a flowing or stand-still, in-line aqueous base fluid sensor assembly/subsystem,
receiving aqueous fluid data from the flowing or stand-still, in-line aqueous base fluid sensor assembly/subsystem via the control assembly/subsystem,
receiving downhole data via the control assembly/subsystem,
adding amounts of downhole fluid components to the aqueous base fluid based the aqueous fluid data via a downhole treating fluid component supply assembly/subsystem to form a downhole treating fluid,
adding amounts of friction-reducing components to the downhole fluid via a friction-reducing component supply assembly/subsystem to form an optimized downhole treating fluid,
injecting the optimized downhole treating fluid via a downhole fluid injection assembly/subsystem into a subterranean formation,
controlling the adding steps and the injecting step via a control assembly/subsystem, and repeating the above step until the treating operation stops.

Embodiment 16. The Embodiment 15, wherein the stand-still or flowing in-line aqueous fluid sensor subsystem is configured to generate real-time or near real-time data concerning a composition and properties of the aqueous base fluid and downhole sensor subsystem is configured to generate real-time or near real-time data concerning a composition and properties of the downhole treating fluid and real-time or near real-time data concerning the treating properties.

Embodiment 17. The Embodiments 15 through 16, wherein the downhole fluid component supply subsystem is configured to supply downhole fluid components to the aqueous base fluid based on the data to form a downhole treating fluid and the friction-reducing component supply subsystem supplies friction-reducing components to the downhole treating fluid based on the data to form an optimized downhole treating fluid having a reduced or minimized percent drag reduction (% DR) value. In certain embodiments, the downhole sensor subsystem may also include a program configured to generate modeling data for adjusting a composition and properties of the optimized downhole treating fluid based on the sensor and modeling data.

Embodiment 18. The Embodiments 15 through 17, wherein the control subsystem is configured to utilize the data to adjust amount of the downhole treating fluid components, the friction-reducing components being added to the aqueous base fluid in real-time or near real-time.

Embodiment 19. A method comprising:
supplying an aqueous base fluid from an aqueous based fluid supply assembly/subsystem,
passing the aqueous base fluid through a flowing or stand-still, in-line aqueous base fluid sensor assembly/subsystem,
receiving aqueous fluid data from the flowing or stand-still, in-line aqueous base fluid sensor assembly/subsystem via the control assembly/subsystem,
receiving downhole data via the control assembly/subsystem,
adding amounts of downhole fluid components to the aqueous base fluid based the aqueous fluid data via a downhole fluid component supply assembly/subsystem to form a downhole drilling fluid,
adding amounts of friction-reducing components to the downhole fluid via a friction-reducing component supply assembly/subsystem to form an optimized downhole drilling fluid,
circulating the optimized downhole drilling fluid via a downhole fluid circulating assembly/subsystem in a wellbore to a subterranean formation,
controlling the adding steps and the circulating step via a control assembly/subsystem, and repeating the above step until the drilling operation stops.

Embodiment 20. The Embodiment 19, wherein the stand-still or flowing in-line aqueous fluid sensor subsystem is configured to generate real-time or near real-time data concerning a composition and properties of the aqueous base fluid and downhole sensor subsystem is configured to generate real-time or near real-time data concerning a composition and properties of the downhole drilling fluid and real-time or near real-time data concerning the drilling properties.

Embodiment 21. The Embodiments 19 through 20, wherein the downhole fluid component supply subsystem is configured to supply downhole fluid components to the aqueous base fluid based on the data to form a downhole fluid and the friction-reducing component supply subsystem supplies friction-reducing components to the downhole drilling fluid based on the data to form an optimized downhole drilling fluid having a reduced or minimized percent drag reduction (% DR) value and the downhole sensor subsystem may also include a program configured to generate modeling data for adjusting a composition and properties of the optimized downhole fluid based on the sensor and modeling data.

Embodiment 22. The Embodiments 19 through 21, wherein the control subsystem is configured to utilize the data to adjust amount of the downhole fluid components, the friction-reducing components being added to the aqueous base fluid in real-time or near real-time.

Embodiment 23. The Embodiments of any preceding Embodiments, wherein the sensors include any sensor that measure a property of water such as salinity sensors, conductivity sensors, pH sensors, or other water properties or specific ion or chemical sensors that measure the concentration of specific ions or chemicals such as sodium ion sensors, potassium ion sensors, carbonate ion sensors, hydronium ion sensors, hydroxide ion sensors, borate ion sensors, carbon dioxide sensors, oxygen sensors, calcium ion sensors, magnesium ion sensors, titanium ion sensors, zirconium ion sensors, other ion specific sensors, other chemical specific sensors, or any combination thereof.

Embodiment 24. The Embodiments of any preceding Embodiments, wherein the aqueous base fluid properties include salinity, conductivity, pH, specific metal ions and/or metal salts, concentrations of the specific metal ions and/or metal salts, other ions and/or chemicals, ionicity, any other property of the aqueous base fluid, or any combination thereof.

Embodiment 25. The Embodiments of any preceding Embodiments, wherein the formation properties include formation temperature or temperature profile, formation pressure or pressure profile, formation geological structural properties, e.g., type of rock, shale, sand, etc., type and nature of natural fractures within the formation, extent of the formation to be treated, depth of penetration of the treating fluid, desired treating results, type of proppants to be used, type of proppant pillar formation, type of pumping format, pumping conditions such as pumping pressure, downhole fluid flow rate, pumping sequences, etc., other formation properties, or any combination thereof.

Embodiment 26. The Embodiments of any preceding Embodiments, wherein the injection equipment and circulation equipment include any injecting and circulating systems used in the art.

Embodiment 27. The Embodiments of any preceding Embodiments, wherein the friction-reducing components comprise friction-reducing polymers include an acrylamide containing polymers or polyacrylamide containing polymers including more than 50% of acrylamide monomer in the polymer backbone.

Embodiment 28. The Embodiment 27, wherein the friction-reducing polymers include at least 30% acrylamide, at least 40% acrylamide, at least 40% acrylamide, at least 50% acrylamide, at least 60% acrylamide, at least 70% acrylamide, at least 80% acrylamide, at least 90% acrylamide, or 100% acrylamide. It should be recognized that these ranges include all subranges such as 30% to 100% or any other range or any other at least percentage.

Embodiment 29. The Embodiments of any preceding Embodiments, wherein the aqueous base fluids include a high TDS produced water, a high TDS flow back water, a high TDS fracturing flow back water, a brackish water, a reverse osmosis (RO) reject water, a clear brine, and mixtures thereof. In certain embodiments, the aqueous base fluids further include fresh water.

Embodiment 30. The Embodiments of any preceding Embodiments, wherein the oil-based base fluids include a hydrocarbon fluid such as diesel, kerosene, fuel oil, selected crude oils, a mineral oil, or any combination thereof.

Embodiment 31. The Embodiment 30, wherein the hydratable polymers or gelling agents include any hydratable polysaccharides that are capable of forming a gel in the presence of a crosslinking agent.

Embodiment 32. The Embodiment 31, wherein the hydratable polysaccharides include galactomannan gums, glucomannan gums, guars, derivatized guars, cellulose derivatives, guar gum derivatives, locust bean gum, Karaya gum, carboxymethyl cellulose, carboxymethyl hydroxyethyl cellulose, and hydroxyethyl cellulose, or mixtures or combinations thereof.

Embodiment 33. The Embodiment 32, wherein the hydratable polysaccharides include guar gums, hydroxypropyl guar, carboxymethyl hydroxypropyl guar, carboxymethyl guar, and carboxymethyl hydroxyethyl cellulose. Suitable hydratable polymers may also include synthetic polymers, such as polyvinyl alcohol, polyacrylamides, poly-2-amino-2-methyl propane sulfonic acid, and various other synthetic polymers and copolymers.

Embodiment 34. The Embodiment 33, wherein the hydratable polysaccharides include the molecular weight of the hydratable synthetic polymers are between about 10,000 to about 100,000,000. In other embodiments, the molecular weight is between about 10,000 to about 10,000,000. In other embodiments, the molecular weight is between about 10,000 to about 1,000,000.

Embodiment 35. The Embodiment 34, wherein the hydratable polymer may be present in a treating or drilling fluid in a polymer concentration ranging from about 0.05 wt. % to about 10 wt. %.

Embodiment 36. The Embodiment 35, wherein the polymer concentration ranges 5 between about 0.10 wt. % and about 5.0 wt. %.

Embodiment 37. The Embodiment 36, wherein the polymer concentration ranges between about 0.05 w. % and about 0.7 wt. % of the aqueous fluid.

Embodiment 38. The Embodiment 37, wherein the polymer concentration ranges between about 0.10 wt. % and about 0.25 wt. %.

Embodiment 39. The Embodiment 38, wherein if the polymer is in the form or a slurry, then the slurry is present in an amount between about 10 gpt and about 30 gpt (gallons per thousand gallons) of the fracturing fluid.

Embodiment 40. The Embodiment 39, wherein the polymer slurry amount is between about 1 gpt and about 15 gpt.

Embodiment 41. The Embodiment 40, wherein the polymer slurry amount is between about between about 2 gpt and about 5 gpt.

Embodiment 42. The Embodiments of any preceding Embodiments, wherein the crosslinking agents include any compound that increases the viscosity of a fluid including the hydratable polymers by chemical crosslinks, physical crosslinks, and/or cross-links the hydratable polymer by any other mechanism.

Embodiment 43. The Embodiment 42, wherein the cross-linking agent includes a metal containing compound.

Embodiment 44. The Embodiment 43, wherein the cross-linking agent includes boron, zirconium, and titanium containing compounds, or mixtures thereof.

Embodiment 45. The Embodiment 44, wherein the cross-linking agent includes an organotitanate compound.

Embodiment 46. The Embodiment 45, wherein the cross-linking agent includes a borate.

Embodiment 47. The Embodiment 46, wherein the selection of an appropriate crosslinking agent depends upon the type of treatment to be performed and the hydratable polymer to be used.

Embodiment 48. The Embodiment 47, wherein an amount of the crosslinking agent used also depends upon the well conditions and the type of treatment to be introduced.

Embodiment 49. The Embodiment 48, wherein the amount of the crosslinking agent ranges from about 10 ppm to about 1000 ppm of metal ion of the crosslinking agent in the hydratable polymer fluid.

Embodiment 50. The Embodiment 49, wherein the crosslinking agent include borate-containing compounds, titanate-containing compounds, zirconium-containing compound, or mixtures thereof.

Embodiment 51. The Embodiment 50, wherein the crosslinking agent includes sodium borate×H₂O (varying waters of hydration), boric acid, a borate, a mixture of a titanium-containing compound and a boron-containing compound, an organotitanate compound, a mixture of a first organotitanate compound having a lactate base and a second organotitanate compound having triethanolamine base, sodium tetraborate, ulexite, colemanite, Ti(IV) acetylacetonate, Ti(IV) triethanolamine, Zr lactate, Zr triethanolamine, Zr lactate-triethanolamine, Zr lactate-triethanolamine-triisopropanolamine, and mixtures thereof.

Embodiment 52. The Embodiment 51, wherein the crosslinking agent includes titanium crosslinking agents, chromium crosslinking agents, iron crosslinking agents, aluminum crosslinking agents, zirconium crosslinking agents, zirconium triethanolamine complexes, zirconium acetylacetonate, zirconium lactate, zirconium carbonate, and chelants of organic alphahydroxycorboxylic acid and zirconium, titanium triethanolamine complexes, titanium acetylacetonate, titanium lactate, and chelants of organic alphahydroxycorboxylic acid and titanium, or combinations thereof.

Embodiment 53. The Embodiments of any preceding Embodiments, wherein the propping agents or proppants include quartz sand grains, glass beads, ceramic beads, coated glass or ceramic beads, walnut shell fragments, aluminum pellets, nylon pellets, or mixtures thereof.

Embodiment 54. The Embodiment 53, wherein the proppants are used in concentrations between about 1 lb to about 8 lbs. per gallon of a fracturing fluid, although higher or lower concentrations may also be used as desired.

Embodiment 55. The Embodiment 54, wherein the fracturing fluid may also contain other additives, such as surfactants, corrosion inhibitors, mutual solvents, stabilizers, paraffin inhibitors, tracers to monitor fluid flow back, and so on.

Embodiment 56. The Embodiments of any preceding Embodiments, wherein the inorganic acids include any inorganic acid.

Embodiment 57. The Embodiment 56, wherein the inorganic acids include hydrogen chloride, sulfuric acid, phosphoric acid, or mixtures thereof.

Embodiment 58. The Embodiments of any preceding Embodiments, wherein the organic acids include any organic acid.

Embodiment 59. The Embodiment 58, wherein the organic acids include formic acid, acetic acid, propionic acid, or mixtures thereof.

Embodiment 60. The Embodiments of any preceding Embodiments, wherein the inorganic bases include any inorganic base.

Embodiment 61. The Embodiment 60, wherein the inorganic bases include sodium hydroxide, sodium bicarbonate, sodium carbonate, sodium bicarbonate, potassium hydroxide, potassium bicarbonate, potassium carbonate, or mixtures thereof.

### Organic Bases

Embodiment 62. The Embodiments of any preceding Embodiments, wherein the organic acids include any organic base.

Embodiment 63. The Embodiment 62, wherein the organic acids include sodium tert-butoxide, potassium tert-butoxide, choline hydroxide, or mixtures thereof.

Embodiment 64. The Embodiments of any preceding Embodiments, wherein the friction-reducing polymers include one or more anionic polymers, one or more cationic polymers, one or more amphoteric polymers, or any combination thereof.

Embodiment 65. The Embodiment 64, wherein the friction-reducing polymers include one or more acrylamide copolymers, one or more anionic acrylamide copolymers, one or more cationic acrylamide copolymers, one or more nonionic acrylamide copolymers, one or more amphoteric acrylamide copolymers, one or more polyacrylamides, one or more polyacrylamide derivatives, one or more polyacrylate, one or more polyacrylate derivative, one or more polymethacrylate, one or more polymethacrylate derivatives, and any mixture or combination thereof.

Embodiment 66. The Embodiment 65, wherein the friction-reducing polymers include polyacrylates, polyacrylate derivatives, polyacrylate copolymers, polymethacrylates, polymethacrylate derivatives, polymethacrylate copolymers, polyacrylamide, polyacrylamide derivatives, polyacrylamide copolymers, acrylamide copolymers, polysaccharides, polysaccharide derivatives, polysaccharide copolymers, synthetic polymers, superabsorbent polymers, and any combination thereof.

Embodiment 67. The Embodiment 66, wherein the friction-reducing polymers include one or more water soluble FR polymers.

Embodiment 68. The Embodiment 67, wherein the one or more water soluble FR polymers include polymers containing one or more of the following monomers: acrylamide, acrylic acid, methacrylic acid, vinyl acetate, vinyl sulfonic acid, N-vinyl acetamide, N-vinyl formamide, itaconic acid, acrylic acid ester, methacrylic acid ester, ethoxylated-2-hydroxyethyl acrylate, ethoxylated-2-hydroxyethyl methacrylate, 2-hydroxyethyl acrylate, 2-hydroxyethylmethyl methacrylate, 2-hydroxypropyl acrylate, 2-hydroxypropyl methacrylate, hydroxymethyl styrene, 2-acrylamido-2-methylpropane sulfonic acid (AMPS), acrylamido tertiary butyl sulfonic acid (ATBS), 2-(meth)acrylamido-2-methylpropane sulfonic acid, 2-amino-2-methyl-1-propanol (AMP), N,N-dimethylacrylamide (DMAA), a salt of any of the foregoing, and any combination thereof.

Embodiment 69. The Embodiment 63, wherein the friction-reducing polymers include one or more copolymers including acrylamide and AMPS.

Embodiment 70. The Embodiment 69, wherein the friction-reducing polymers include high molecular weight, linear polymers.

Embodiment 71. The Embodiment 70, wherein the friction-reducing polymers include friction reducing polymers include one or more monomers selected from the groups consisting of acrylamide, acrylic acid, 2-acrylamido-2-methylpropane sulfonic acid, acrylamido tertiary butyl sulfonic acid, a salt of any of the foregoing, or any mixture or combination thereof.

Embodiment 72. The Embodiment 71, wherein the friction-reducing polymers have molecular weights ranging from about 100,000 to about 40,000,000, from about 200,000 to about 35,000,000, from about 300,000 to about 30,000,000, from about 400,000 to about 25,000,000, or from about 500,000 to about 20,000,000.

Embodiment 73. The Embodiments of any preceding Embodiments, wherein the hydration delaying salt compositions include ammonium sulfate or a mixture of ammonium sulfate and one or more other salts, one or more carbonate salts, one or more sulfate salts, one or more phosphate salts, one or more magnesium salts, one or more bromide salts, one or more formate salts, one or more acetate salts, one or more chloride salts, one or more fluoride salts, a bicarbonate salts, one or more nitrate salts, and any mixture or combination thereof.

Embodiment 74. The Embodiment 73, wherein the one or more carbonate salts include ammonium carbonate, sodium carbonate, potassium carbonate, aluminum carbonate, magnesium carbonate, calcium carbonate, barium carbonate, strontium carbonate, zinc carbonate, other metal carbonates, or any mixture or combination thereof.

Embodiment 75. The Embodiment 74, wherein the one or more phosphate salts include ammonium sulfate, sodium sulfate, potassium sulfate, aluminum sulfate, magnesium sulfate, calcium sulfate, barium sulfate, strontium sulfate, zinc sulfate, other metal sulfates, or any mixture or combination thereof.

Embodiment 76. The Embodiment 75, wherein the one or more chloride salts include ammonium chloride, sodium chloride, potassium chloride, calcium chloride, magnesium chloride, strontium chloride, barium chloride, other metal chlorides, or any mixture or combination thereof.

Embodiment 77. The Embodiment 76, wherein the one or more bromide salts include sodium bromide, potassium bromide, calcium bromide, magnesium bromide, zinc bromide, strontium bromide, other metal bromides, or any mixture or combination thereof.

Embodiment 78. The Embodiment 77, wherein the one or more bicarbonates include sodium bicarbonate, potassium bicarbonate, other metal bicarbonates, or any mixture or combination thereof.

Embodiment 79. The Embodiment 78, wherein the one or more nitrate salts include sodium nitrate, potassium nitrate, calcium nitrate, magnesium nitrate, zinc nitrate, strontium nitrate, other metal nitrate, or any mixture or combination thereof.

Embodiment 80. The Embodiment 79, wherein the hydration delaying salt compositions include divalent salts such as calcium and/or magnesium salts and monovalent salts such as ammonium and/or potassium salts work to prevent hydration as well.

Embodiment 81. The Embodiment 80, wherein the hydration delaying salt compositions include phosphate based salts such as potassium phosphate and/or variants such as potassium hexametaphosphate, which are capable of delaying or preventing FR polymer hydration.

Embodiment 82. The Embodiment 81, wherein the hydration delaying salt compositions include water-soluble potassium salts selected from the group consisting of potassium citrate, potassium carbonate, or mixtures thereof.

Embodiment 83. The Embodiment 82, wherein the hydration delaying salt compositions include multivalent salts such as zinc chloride, aluminum chloride, iron chloride, zinc sulfate, aluminum sulfate, iron sulfate, or any combination thereof.

Embodiment 84. The Embodiment 83, wherein the hydration delaying salt compositions include double salt equivalents such as magnesium ammonium sulfates, calcium ammonium sulfates, aluminum ammonium sulfates, iron ammonium sulfates, nickel ammonium sulfates, copper ammonium sulfates, similar metal ammonium salts, or any combination thereof.

Embodiment 85. The Embodiments of any preceding Embodiments, wherein the suspending agents include a bentonite clay, a phyllosilicate clay, nano-structured clays, micro-structured clay, or any combination thereof.

Embodiment 86. The Embodiments of any preceding Embodiments, wherein the gel-bridging agents polyethylene glycols such as PEG 200, PEG 300, PEG 400, PEG 500, or similar polyethylene polymers, polypropylene glycols, polyethylene/propylene glycols, other polyalkylene oxide polymers, or any mixture thereof.

Embodiment 87. The Embodiments of any preceding Embodiments, wherein the pH adjusting agents include organic acids selected from the group consisting of fatty acid, diacids, polyacids, citric acid, oxalic acid, ascorbic acid, isocitric acid, aconitic acid, propane-1,2,3-tricarboxylic acid, and any mixture thereof.

Embodiment 88. The Embodiment 87, wherein the fatty acids include any saturated fatty acid or unsaturated fatty acids or mixtures or combinations thereof.

Embodiment 89. The Embodiment 88, wherein the fatty acids include short chain free fatty acids (SCFFA), medium chain free fatty acids (MCFFA), long chain free fatty acids (LCFFA), very-long-chain free fatty acids (VLCFFA), or mixtures or combinations thereof.

Embodiment 90. The Embodiment 89, wherein the SCFFA include free fatty acids having a carbyl tail group having less than between 4 and less than 8 carbon atoms (C₄ to C₈).

Embodiment 91. The Embodiment 90, wherein the MCFFA include free fatty acids having a carbyl group having between 8 and less than 14 carbon atoms (C₈ to C₁₄).

Embodiment 92. The Embodiment 91, wherein the LCFFA include free fatty acids having a carbyl group having between 14 and 24 carbon atoms (C₁₄-C₂₄).

Embodiment 93. The Embodiment 92, wherein the VLCFFA include free fatty acids having a carbyl group having greater than 24 carbon atoms (>C₂₄).

Embodiment 94. The Embodiment 93, wherein the unsaturated fatty acids include myristoleic acid [CH₃(CH₂)₃CH═CH(CH₂)₇COOH, cis-Δ⁹, C:D 14:1, n-5], palmitoleic acid [CH₃(CH₂)₅CH═CH(CH₂), COOH, cis-Δ⁹, C:D 16:1, n-7], sapienic acid [CH₃(CH₂)₈CH═CH(CH₂)₄COOH, cis-Δ⁶, C:D 16:1, n-10], oleic acid [CH₃(CH₂)₇CH═CH(CH₂)₇COOH, cis-Δ⁹, C:D 18:1, n-9], linoleic acid [CH₃(CH₂)₄CH═CHCH₂CH═CH(CH₂)₇COOH, cis, cis-Δ⁹,Δ¹², C:D 18:2, n-6], α-Linolenic acid [CH₃CH₂CH═CHCH₂CH═CHCH₂CH═CH(CH₂)₇COOH, cis, cis, cis-Δ⁹,Δ¹²,Δ¹⁵, C:D 18:3, n-3], arachidonic acid [CH₃(CH₂)₄CH═CHCH₂CH═CHCH₂CH═CHCH₂CH═CH(CH₂)₃COOH, cis, cis, cis, cis-Δ⁵Δ⁸, Δ¹¹, Δ¹⁴, C:D 20:4, n-6], cicosapentaenoic acid [CH₃CH₂CH═CHCH₂CH═CHCH₂CH═CHCH₂CH═CHCH₂CH═CH(CH₂)₃COOH], cis, cis, cis, cis, cis-Δ⁵,Δ⁸,Δ¹¹,Δ¹⁴,Δ¹⁷, 20:5, n-3], erucic acid [CH₃(CH₂)₇CH═CH(CH₂)₁₁COOH, cis-Δ¹³, C:D 22:1, n-9], docosahexaenoic acid [CH₃CH₂CH═CHCH₂CH═CHCH₂CH═CHCH₂CH═CH CH₂CH═CHCH₂CH═CH(CH₂)₂COOH, cis,cis, cis, cis, cis, cis-Δ⁴,Δ⁷,Δ¹⁰,Δ¹³,Δ¹⁶,Δ¹¹, C:D 22:6, n-3], or mixtures and combinations thereof.

Embodiment 95. The Embodiment 94, wherein the saturated fatty acids include lauric acid [CH₃(CH₂)₁₀COOH, C:D 12:0], myristic acid [CH₃(CH₂)₁₂COOH, C:D 14:0], palmitic acid [CH₃(CH₂)₁₄COOH, C:D 16:0], stearic acid [CH₃(CH₂)₁₆COOH, C:D 18:0], arachidic acid [CH₃(CH₂)₁₈COOH, C:D 20:0], behenic acid [CH₃(CH₂)₂₀COOH, C:D 22:0], lignoceric acid [H₃(CH₂)₂₂COOH, C:D 24:0], cerotic acid [CH₃(CH₂)₂₄COOH, C:D 26:0], or mixture or combinations thereof.

Embodiment 96. The Embodiment 95, wherein the saturated fatty acids include butyric (C₄), valeric (C₅), caproic (C₆), enanthic (C₇), caprylic (C₈), pelargonic (C₉), capric (C₁₀), undecylic (C₁₁), lauric (C₁₂), tridecylic (C₁₃), myristic (C₁₄), pentadecylic (C₁₅), palmitic (C₁₆), margaric (C₁₇), stearic (C₁₈), nonadecylic (C₁₉), arachidic (C₂₀), heneicosylic (C₂₁), behenic (C₂₂), tricosylic (C₂₃), lignoceric (C₂₄), pentacosylic (C₂₅), cerotic (C₂₆), heptacosylic (C₂₇), montanic (C₂₈), nonacosylic (C₂₉), melissic (C₃₀), hentriacontylic (C₃₁), lacceroic (C₃₂), psyllic (C₃₃), geddic (C₃₄), ceroplastic (C₃₅), hexatriacontylic (C₃₆), heptatriacontylic acid (C₃₇), octatriacontylic acid (C₃₈), nonatriacontylic acid (C₃₉), tetracontylic acid (C₄₀), and mixtures or combinations thereof.

Embodiment 97. The Embodiment 96, wherein the fatty acids include n-3 unsaturated fatty acids such as α-linolenic acid, stearidonic acid, eicosapentaenoic acid, and docosahexaenoic acid, n-6 unsaturated fatty acids such as linoleic acid, γ-linolenic acid, dihomo-γ-linolenic acid, and arachidonic acid, n-9 unsaturated fatty acids oleic acid, claidic acid, cicosenoic acid, erucic acid, nervonic acid, mead acid or mixtures or combinations thereof.

Embodiment 98. The Embodiment 97, wherein the unsaturated fatty acids include (a) ω-3 unsaturated fatty acids such as octenoic (8:1), decenoic (10:1), decadienoic (10:2), lauroleic (12:1), laurolinoleic (12:2), myristovaccenic (14:1), myristolinoleic (14:2), myristolinolenic (14:3), palmitolinolenic (16:3), palmitidonic (16:4), α-linolenic (18:3), stearidonic (18:4), dihomo-α-linolenic (20:3), cicosatetraenoic (20:4), cicosapentaenoic (20:5), clupanodonic (22:5), docosahexaenoic (22:6), 9, 12, 15, 18, 21-tetracosapentaenoic (24:5), 6,9,12,15,18,21-tetracosahexaenoic (24:6), and mixtures or combinations thereof; (b) ω-5 unsaturated such as myristoleic (14:1), palmitovaccenic (16:1), α-eleostearic (18:3), β-eleostearic (trans-18:3) punicic (18:3), 7,10,13-octadecatrienoic (18:3), 9,12,15-cicosatrienoic (20:3), β-eicosatetraenoic (20:4), and mixtures or combinations thereof; (c) ω-6 unsaturated such as 8-tetradecenoic (14:1), 12-octadecenoic (18:1), linoleic (18:2), linolelaidic (trans-18:2), γ-linolenic (18:3), calendic (18:3), pinolenic (18:3), dihomo-linoleic (20:2), dihomo-γ-linolenic (20:3), arachidonic (20:4), adrenic (22:4), osbond (22:5), and mixtures or combinations thereof; (d) ω-7 unsaturated such as palmitoleic (16:1), vaccenic (18:1), rumenic (18:2), paullinic (20:1), 7,10,13-cicosatrienoic (20:3), and mixtures or combinations thereof; (c) ω-9 Unsaturated such as oleic (18:1), claidic (trans-18:1), gondoic (20:1), erucic (22:1), nervonic (24:1), 8,11-eicosadienoic (20:2), mead (20:3), and mixtures or combinations thereof; (f) ω-10 Unsaturated such as Sapienic (16:1); (g) ω-11 unsaturated such as gadoleic (20:1); (h) ω-12 Unsaturated such as 4-Hexadecenoic (16:1) Petroselinic (18:1) 8-Eicosenoic (20:1), and mixtures or combinations thereof; and (i) mixtures or combinations thereof.

Embodiment 99. The Embodiment 98, wherein the saturate diacids include ethanedioic acid (oxalic acid), propanedioic acid (malonic acid), butanedioic acid (succinic acid), pentanedioic acid (glutaric acid), hexanedioic acid (adipic acid), heptanedioic acid (pimelic acid), octanedioic acid (suberic acid, nonanedioic acid (azelaic acid), decanedioic acid (sebacic acid), undecanedioic acid, dodecanedioic acid, tridecanedioic acid (brassylic acid), hexadecanedioic acid (thapsic acid), heneicosa-1,21-dioic acid (japanic acid), docosanedioic acid (phellogenic acid), triacontanedioic acid (equisetolic acid), or mixtures or combinations thereof.

Embodiment 100. The Embodiment 99, wherein the unsaturated diacids include (Z)-butenedioic acid (maleic acid), (E)-butenedioic acid (fumaric acid), (Z and E)-pent-2-enedioic acid (glutaconic acid), 2-decenedioic acid, dodec-2-enedioic acid (traumatic acid), (2E,4E)-hexa-2,4-dienedioic acid (muconic acid), or mixtures or combinations thereof.

Embodiment 101. The Embodiment 100, wherein the poly carboxylic acid compounds for use a pH depending release agents include any poly carboxylic acid compound.

Embodiment 102. The Embodiment 101, wherein the water immiscible poly acids include dicarboxylic acids having carbyl or carbenyl groups having between 8 and 50 carbon atoms and mixtures or combinations thereof.

Embodiment 103. The Embodiment 102, wherein the polymer carboxylic acids or polymers including carboxylic acid groups, where the polymers are oil soluble or are oils, not miscible with water.

Embodiment 104. The Embodiment 103, wherein the hydrophilic poly acids include polyacrylic acid, polymethacrylic acid, polylactic acid, polyglycol acid, mixtures and combinations thereof, copolymers thereof, CARBOPOLR reagents available from Lubrizol Corporation (a registered trademark of the Lubrizol Corporation), other carboxylic acid containing polymers, or mixtures or combinations thereof.

Embodiment 105. The Embodiment 104, wherein the hydroxy acids include 2-hydroxyoleic acid, 2-hydroxytetracosanoic acid (cerebronic acid), 2-hydroxy-15-tetracosenoic acid (hydroxynervonic acid), 2-hydroxy-9-cis-octadecenoic acid, 3-hydroxypalmitic acid methyl ester, 2-hydroxy palmitic acid, 10-hydroxy-2-decenoic acid, 12-hydroxy-9-octadecenoic acid (ricinoleic acid), 1,13-dihydroxy-tetracos-9t-enoic acid (axillarenic acid), 3,7-dihydroxy-docosanoic acid (byrsonic acid), 9,10-dihydroxyoctadecanoic acid, 9,14-dihydroxyoctadecanoic acid, 22-hydroxydocosanoic acid (phellonic acid), 2-oxo-5,8,12-trihydroxydodecanoic acid (phaseolic acid), 9,10,18-trihydroxyoctadecanoic acid (phloionolic acid), 7,14-dihydroxydocosa-4Z, 8,10,12,16Z,19Z-hexaenoic acid (Maresin 1), 5S, 12R, 18R-trihydroxy-6Z,8E,10E,14Z,16E-eicosapentaenoic acid (resolvin E1), resolvin D1, 10,17S-docosatriene, (neuroprotectin D1).

Embodiment 106. The Embodiments of any preceding Embodiments, wherein the treating fluid additives include proppants, acids, diverting agents, fluid loss control additives, gas, nitrogen, carbon dioxide, surface modifying agents, tackifying agents, foamers, corrosion inhibitors, scale inhibitors, catalysts, clay control agents, biocides, antifoam agents, bridging agents, flocculants, H₂S scavengers, CO₂ scavengers, oxygen scavengers, lubricants, viscosifiers, breakers, weighting agents, relative permeability modifiers, resins, surfactants, wetting agents, coating enhancement agents, filter cake removal agents, antifreeze agents, ethylene glycol, or mixtures thereof.

Embodiment 107. The Embodiments of any preceding Embodiments, wherein the nonionic and amphoteric polymers used in the present composition preferably exhibit a molecular weight within the range of about 8 million to about 14 million or ranging from about 10 million to 15 million or ranging from about 10 million to about 12 million.

Embodiment 108. The Embodiment 107, wherein the anionic, cationic, or amphoteric polymers include homopolymers, copolymers, terpolymers, or high order mixed monomer polymers synthesized from one or more anionic monomers, cationic monomers, and/or neutral monomers.

Embodiment 109. The Embodiment 108, wherein the copolymer and high order mixed monomer polymers, the monomers used may have similar reactivities so that the resultant amphoteric polymeric material has a random distribution of monomers.

Embodiment 110. The Embodiment 109, wherein the anionic monomers may be any anionic monomer such as acrylic acid, methacrylic acid, 2-acrylamide-2-methylpropane sulfonic acid, maleic anhydride, or any combination thereof.

Embodiment 111. The Embodiment 110, wherein the cationic monomer includes dimethyl-diallyl ammonium chloride, dimethylamino-ethyl methacrylate, allyltrimethyl ammonium chloride, or any combination thereof.

Embodiment 112. The Embodiment 111, wherein the neutral monomer includes butadiene, N-vinyl-2-pyrrolidone, methyl vinyl ether, methyl acrylate, maleic anhydride, styrene, vinyl acetate, acrylamide, methyl methacrylate, and/or acrylonitrile.

Embodiment 113. The Embodiment 112, wherein the polymers include a terpolymer synthesized from acrylic acid (AA), dimethyl diallyl ammonium chloride (DMDAC) or diallyl dimethyl ammonium chloride (DADMAC), and acrylamide (AM) having any ratio of monomers in the terpolymer includes a 1:1:1 ratio.

Embodiment 114. The Embodiment 113, wherein the amphoteric polymeric materials include about 30% polymerized AA, 40% polymerized AM, and 10% polymerized DMDAC or DADMAC with about 20% free residual DMDAC or DADMAC which is not polymerized due to lower relative reactivity of the DMDAC or DADMAC monomer.

### CLOSING PARAGRAPH OF THE DISCLOSURE

Therefore, the present disclosure is well adapted to attain the ends and advantages mentioned as well as those that are inherent therein. The embodiments disclosed above are illustrative only, as the present disclosure may be modified and practiced in different but equivalent manners apparent to those skilled in the art having the benefit of the teachings herein. While numerous changes may be made by those skilled in the art, such changes are encompassed within the spirit of the subject matter defined by the appended claims. Furthermore, no limitations are intended to the details of construction or design herein shown, other than as described in the claims below. It is therefore evident that the particular illustrative embodiments disclosed above may be altered or modified and all such variations are considered within the scope and spirit of the present disclosure. All references cited herein are incorporated by reference. Although the disclosure has been disclosed with reference to its preferred embodiments, from reading this description those of skill in the art may appreciate changes and modification that may be made which do not depart from the scope and spirit of the disclosure as described above and claimed hereafter.

1. An apparatus comprising:
an aqueous base fluid supply assembly,
a flowing in-line aqueous fluid sensor assembly or a stand-still in-line aqueous fluid sensor assembly,
a downhole sensor assembly,
a downhole fluid component supply assembly,
a friction-reducing component supply assembly,
a control assembly,
wherein the apparatus is configured to adjust in real-time or near real-time, a composition of the downhole fluid based on data received from the in-line sensor assembly.

2. The apparatus of claim 1, wherein:
the stand-still or flowing in-line aqueous fluid sensor assembly is configured to generate real-time or near real-time data concerning a composition and properties of the aqueous base fluid and downhole sensor assembly is configured to generate real-time or near real-time data concerning a composition and properties of the downhole fluid and real-time or near real-time data concerning the treating or drilling properties;
the downhole fluid component supply assembly is configured to supply downhole fluid components to the aqueous base fluid based on the data to form a downhole fluid and the friction-reducing component supply assembly supplies friction-reducing components to the downhole fluid based on the data to form an optimized downhole fluid having a reduced or minimized percent drag reduction (% DR) value. In certain embodiments, the downhole sensor assembly may also include a program configured to generate modeling data for adjusting a composition and properties of the optimized downhole fluid based on the sensor and modeling data; and
the control assembly is configured to utilize the data to adjust amount of the downhole fluid components, the friction-reducing components being added to the aqueous base fluid in real-time or near real-time.

3. The apparatus of claim 1, further comprising:
a downhole fluid injecting assembly when the downhole fluid is a treating fluid, or a downhole fluid circulating assembly when the downhole fluid is a drilling fluid.

4. The apparatus of claim 3, wherein:
the stand-still or flowing in-line aqueous fluid sensor assembly is configured to generate real-time or near real-time data concerning a composition and properties of the aqueous base fluid and downhole sensor assembly is configured to generate real-time or near real-time data concerning a composition and properties of the downhole fluid and real-time or near real-time data concerning the treating or drilling properties;
the downhole fluid component supply assembly is configured to supply downhole fluid components to the aqueous base fluid based on the data to form a downhole fluid and the friction-reducing component supply assembly supplies friction-reducing components to the downhole fluid based on the data to form an optimized downhole fluid having a reduced or minimized percent drag reduction (% DR) value. In certain embodiments, the downhole sensor assembly may also include a program configured to generate modeling data for adjusting a composition and properties of the optimized downhole fluid based on the sensor and modeling data; and
the control assembly is configured to utilize the data to adjust amount of the downhole fluid components, the friction-reducing components being added to the aqueous base fluid in real-time or near real-time.

5. The apparatus of claim 4, wherein the injection equipment and circulation equipment include any injecting and circulating systems used in the art.

6. The apparatus of claim 1, wherein the aqueous base fluids include a high TDS produced water, a high TDS flow back water, a high TDS fracturing flow back water, a brackish water, a reverse osmosis (RO) reject water, a clear brine, and mixtures thereof. In certain embodiments, the aqueous base fluids further include fresh water.

7. The apparatus of claim 1, wherein the friction-reducing polymers include an acrylamide containing polymers or polyacrylamide containing polymers including more than 50% of acrylamide monomer in the polymer backbone.

8. The apparatus of claim 1, wherein the tion-reducing polymers include at least 30% acrylamide, at least 40% acrylamide, at least 40% acrylamide, at least 50% acrylamide, at least 60% acrylamide, at least 70% acrylamide, at least 80% acrylamide, at least 90% acrylamide, or 100% acrylamide. It should be recognized that these ranges include all subranges such as 30% to 100% or any other range or any other at least percentage.

9. The apparatus of claim 1, wherein the hydratable polymers or gelling agents include any hydratable polysaccharides that are capable of forming a gel in the presence of a crosslinking agent.

10. The apparatus of claim 1, wherein the hydratable polysaccharides include galactomannan gums, glucomannan gums, guars, derivatized guars, cellulose derivatives, guar gum derivatives, locust bean gums, Karaya gums, carboxymethyl celluloses, carboxymethyl hydroxyethyl celluloses, hydroxyethyl celluloses, or mixtures or combinations thereof.

11. A method comprising:
supplying an aqueous base fluid from an aqueous based fluid supply assembly/subsystem,
passing the aqueous base fluid through a flowing or stand-still, in-line aqueous base fluid sensor assembly/subsystem,
receiving aqueous fluid data from the flowing or stand-still, in-line aqueous base fluid sensor assembly/subsystem via the control assembly/subsystem,
receiving downhole data via the control assembly/subsystem,
adding amounts of downhole fluid components to the aqueous base fluid based the aqueous fluid data via a downhole fluid component supply assembly/subsystem to form a downhole fluid,
adding amounts of friction-reducing components to the downhole fluid via a friction-reducing component supply assembly/subsystem to form an optimized downhole fluid,
circulating the optimized downhole drilling fluid via a downhole fluid circulating assembly/subsystem in a wellbore to a subterranean formation,
controlling the adding steps and the circulating step via a control assembly/subsystem, and repeating the above step until the drilling operation stops.

12. The method of claim 11, wherein:
the stand-still or flowing in-line aqueous fluid sensor assembly is configured to generate real-time or near real-time data concerning a composition and properties of the aqueous base fluid and downhole sensor assembly is configured to generate real-time or near real-time data concerning a composition and properties of the downhole fluid and real-time or near real-time data concerning the treating or drilling properties;
the downhole fluid component supply assembly is configured to supply downhole fluid components to the aqueous base fluid based on the data to form a downhole fluid and the friction-reducing component supply assembly supplies friction-reducing components to the downhole fluid based on the data to form an optimized downhole fluid having a reduced or minimized percent drag reduction (% DR) value. In certain embodiments, the downhole sensor assembly may also include a program configured to generate modeling data for adjusting a composition and properties of the optimized downhole fluid based on the sensor and modeling data; and
the control assembly is configured to utilize the data to adjust amount of the downhole fluid components, the friction-reducing components being added to the aqueous base fluid in real-time or near real-time.

13. The method of claim 11, further comprising:
a downhole fluid injecting assembly when the downhole fluid is a treating fluid, or
a downhole fluid circulating assembly when the downhole fluid is a drilling fluid.

14. The method of claim 13, wherein:
the stand-still or flowing in-line aqueous fluid sensor assembly is configured to generate real-time or near real-time data concerning a composition and properties of the aqueous base fluid and downhole sensor assembly is configured to generate real-time or near real-time data concerning a composition and properties of the downhole fluid and real-time or near real-time data concerning the treating or drilling properties;
the downhole fluid component supply assembly is configured to supply downhole fluid components to the aqueous base fluid based on the data to form a downhole fluid and the friction-reducing component supply assembly supplies friction-reducing components to the downhole fluid based on the data to form an optimized downhole fluid having a reduced or minimized percent drag reduction (% DR) value. In certain embodiments, the downhole sensor assembly may also include a program configured to generate modeling data for adjusting a composition and properties of the optimized downhole fluid based on the sensor and modeling data; and
the control assembly is configured to utilize the data to adjust amount of the downhole fluid components, the friction-reducing components being added to the aqueous base fluid in real-time or near real-time.

15. The method of claim 14, wherein the injection equipment and circulation equipment include any injecting and circulating systems used in the art.

16. The method of claim 11, wherein the aqueous base fluids include a high TDS produced water, a high TDS flow back water, a high TDS fracturing flow back water, a brackish water, a reverse osmosis (RO) reject water, a clear brine, and mixtures thereof. In certain embodiments, the aqueous base fluids further include fresh water.

17. The method of claim 11, wherein the friction-reducing polymers include an acrylamide containing polymers or polyacrylamide containing polymers including more than 50% of acrylamide monomer in the polymer backbone.

18. The method of claim 11, wherein the tion-reducing polymers include at least 30% acrylamide, at least 40% acrylamide, at least 40% acrylamide, at least 50% acrylamide, at least 60% acrylamide, at least 70% acrylamide, at least 80% acrylamide, at least 90% acrylamide, or 100% acrylamide. It should be recognized that these ranges include all subranges such as 30% to 100% or any other range or any other at least percentage.

19. The method of claim 11, wherein the hydratable polymers or gelling agents include any hydratable polysaccharides that are capable of forming a gel in the presence of a crosslinking agent.

20. The method of claim 11, wherein the hydratable polysaccharides include galactomannan gums, glucomannan gums, guars, derivatized guars, cellulose derivatives, guar gum derivatives, locust bean gums, Karaya gums, carboxymethyl celluloses, carboxymethyl hydroxyethyl celluloses, hydroxyethyl celluloses, or mixtures or combinations thereof.
